SEQUENCE LISTING (18 sequences)

SEQ ID NO: 1 - DNA, 35 bp, Artificial sequence
  Primer oligonucleotide
gctggcacgtccgaacgcttcgttaatccgttgag

SEQ ID NO: 2 - DNA, 35 bp, Artificial sequence
  Primer oligonucleotide
cgtcgtctgccatggcgcttcggtggatatgaact

SEQ ID NO: 3 - DNA, 35 bp, Artificial sequence
  Primer oligonucleotide
acggccgctaatatcaacgcgtcgaatccgcaact

SEQ ID NO: 4 - DNA, 34 bp, Artificial sequence
  Primer oligonucleotide
gccgcgttacgttagccggactattcgatgcagc

SEQ ID NO: 5 - DNA, 37 bp, Artificial sequence
  Primer oligonucleotide
cgaattcactagtgattaatgatacggcgaccaccga

SEQ ID NO: 6 - DNA, 35 bp, Artificial sequence
  Primer oligonucleotide
gcgggaattcgattcaagcagaagacggcatacga

SEQ ID NO: 7 - DNA, 55 bp, Artificial sequence
  Primer oligonucleotide
gctggcacgtccgaacgcttcgttaatccgttgaggatcagctgaagacggtgat

SEQ ID NO: 8 - DNA, 56 bp, Artificial sequence
  Primer oligonucleotide
cgtcgtctgccatggcgcttcggtggatatgaactgatgaaggtatagatatagag

SEQ ID NO: 9 - DNA, 56 bp, Artificial sequence
  Primer oligonucleotide
acggccgctaatatcaacgcgtcgaatccgcaactgatgaaggtatagatatagag

SEQ ID NO: 10 - DNA, 56 bp, Artificial sequence
  Primer oligonucleotide
tgccgcgttacgttagccggactattcgatgcagcgatgaaggtatagatatagag

SEQ ID NO: 11 - DNA, 45 bp, Artificial sequence
  Primer oligonucleotide
ttttttttttgctggcacgtccgaacgcttcgttaatccgttgag

SEQ ID NO: 12 - DNA, 45 bp, Artificial sequence
  Primer oligonucleotide
ttttttttttcgtcgtctgccatggcgcttcggtggatatgaact

SEQ ID NO: 13 - DNA, 45 bp, Artificial sequence
  Oligonucleotide primer
ttttttttttacggccgctaatatcaacgcgtcgaatccgcaact

SEQ ID NO: 14 - DNA, 45 bp, Artificial sequence
  Primer oligonucleotide
tttttttttttgccgcgttacgttagccggactattcgatgcagc

SEQ ID NO: 15 - DNA, 47 bp, Artificial sequence
  Primer oligonucleotide
ttttttttttcgaattcactagtgattaatgatacggcgaccaccga

SEQ ID NO: 16 - DNA, 45 bp, Artificial sequence
  Primer oligonucleotide
ttttttttttgcgggaattcgattcaagcagaagacggcatacga

SEQ ID NO: 17 - DNA, 55 bp, Artificial sequence
  Primer oligonucleotide
cgtcgtctgccatggcgcttcggtggatatgaactgatcagctgaagacggtgat

SEQ ID NO: 18 - DNA, 55 bp, Artificial sequence
  Primer oligonucleotide
acggccgctaatatcaacgcgtcgaatccgcaactgatcagctgaagacggtgat

(19) United States
(12) Patent Application Publication
(10) Pub. No.: US 2009/0117621 A1
(43) Pub. Date: May 7, 2009

(54) Methods of nucleic acid amplification and sequencing

(76) Inventors: Jonathan Mark Boutell, Walden Essex (GB); Geoffrey Paul Smith, Walden (GB); Anthony James Cox, Walden Essex (GB); David James Earnshaw, Walden Essex (GB); Gary Paul Schroth, Walden Essex (GB)

Correspondence Address: KLAUBER & JACKSON, 411 HACKENSACK AVENUE, HACKENSACK, NJ, 07601, US

(21) Appl. No.: 11/989,171

(22) PCT Filed: Jul. 20, 2006
(86) PCT No.: PCT/GB2006/002693

§ 371 Date: Jan. 5, 2009

(30) Foreign Application Priority Data
Jul. 20, 2005 (GB) 0514909.1

(51) Int. Cl. C07H 21/00; C12P 19/34

(52) U.S. Cl. 435/91.2; 536/24.33

(57) ABSTRACT

The invention relates to a method of amplifying one or more nucleic acid templates on a solid support in a nucleic acid amplification reaction, for example by solid-phase PCR using one or more amplification primers attached to the solid support. The method is characterised in that the amplification primers used comprise a template-specific portion which is a sequence of at least 26 consecutive nucleotides and are not capable of annealing to target regions in the template under conditions of the amplification reaction. The method is particularly useful for amplifying human genomic DNA.

### FIELD OF THE INVENTION

The invention relates to methods of nucleic acid amplification and sequencing on a solid support.

### BACKGROUND

Molecular biology and pharmaceutical drug development now make intensive use of nucleic acid analysis. The most challenging areas are whole genome sequencing, single nucleotide polymorphism detection, screening and gene expression monitoring.

One area of technology which has improved the study of nucleic acids is the development of fabricated arrays of immobilised nucleic acids. These arrays typically consist of a high-density matrix of polynucleotides immobilised onto a solid support material. Fodor et al., Trends in Biotechnology (1994) 12:19-26, describe ways of assembling the nucleic acid arrays using a chemically sensitised glass surface protected by a mask, but exposed at defined areas to allow attachment of suitably modified nucleotides. Typically, these arrays may be described as “many molecule” arrays, as distinct regions are formed on the solid support comprising a high density of one specific type of polynucleotide.

An alternative approach is described by Schena et al., Science (1995) 270:467-470, where samples of DNA are positioned at predetermined sites on a glass microscope slide by robotic micropipetting techniques.

Fabricated arrays may also be manufactured by the technique of “spotting” known polynucleotides onto a solid support at predetermined positions (e.g. Stimpson et al PNAS (1995) 92:6379-6383).

WO 98/44151 and WO 00/18957 both describe methods of forming polynucleotide arrays based on “solid-phase” nucleic acid amplification, which is analogous to a polymerase chain reaction wherein the amplification products are immobilised on a solid support in order to form arrays comprised of clusters or “colonies”. Each cluster or colony on such an array is formed from a plurality of identical immobilised polynucleotide strands and a plurality of identical immobilised complementary polynucleotide strands. The arrays so-formed are generally referred to herein as “clustered arrays” and their general features will be further understood by reference to WO 98/44151 or WO 00/18957, the contents of both documents being incorporated herein in their entirety by reference.

As aforesaid, the solid-phase amplification methods of WO 98/44151 and WO 00/18957 are essentially a form of the polymerase chain reaction carried out on a solid support. Like any PCR reaction these methods require the use of forward and reverse amplification primers capable of annealing to the template to be amplified. In the methods of WO 98/44151 and WO 00/18957 both primers are immobilised on the solid support at the 5′ end. Other forms of solid-phase amplification are known in which only one primer is immobilised and the other is present in free solution (Mitra, R. D and Church, G. M., Nucleic Acids Research, 1999, Vol. 27, No. 24). In all PCR-based techniques the forward and reverse amplification primers must include a “template-specific” sequence of nucleotides which is capable of annealing to the template to be amplified, or the complement thereof, under the conditions of the annealing steps of the PCR reaction.

The present invention is based on the finding that the efficiency of solid-phase nucleic acid amplification can be substantially improved by increasing the length of the template-specific portion in the amplification primers beyond the standard length generally used in the prior art. Surprisingly, use of such “long primers” has been observed to substantially improve the efficiency of solid-phase amplification reaction. In the case of clustered arrays, use of such “long primers” increases the efficiency of cluster formation, resulting in clusters which contain significantly more amplified nucleic acid when compared to those produced using the prior art standard primers for the same number of amplification cycles.

The ability to produce clustered arrays containing more nucleic acid per cluster for the same number of amplification cycles is a significant advantage if the arrays are to be used to provide templates for applications involving nucleic acid sequencing.

### SUMMARY OF THE INVENTION

In a first aspect the invention provides a method of amplifying one or more nucleic acid templates on a solid support which comprises:

a) bringing into contact the following components under conditions which permit a nucleic acid amplification reaction:
i) a solid support,
ii) a plurality of forward and reverse amplification primers, wherein the solid support is provided with the forward and/or reverse amplification primers immobilised thereon, and
iii) one or more nucleic acid templates comprising at the 3′ end a sequence of nucleotides capable of annealing to the forward amplification primers and at the 5′ end a sequence of nucleotides the complement of which is capable of annealing to the reverse amplification primers; and
b) carrying out a nucleic acid amplification reaction whereby said template(s) is/are amplified with said forward and reverse amplification primers,
characterised in that the amplification primers immobilised on the solid support comprise a template-specific portion which is a sequence of at least 26 consecutive nucleotides capable of annealing to a primer-binding sequence in the template or the complement thereof and that the forward and reverse primers are not capable of annealing to any part of the template other than their respective primer binding sequences during the nucleic acid amplification reaction.

In one embodiment the one or more templates to be amplified each include a target sequence located between the two primer binding sequences, each said target sequence representing a fragment of the full sequence of a nucleic acid sample of interest. The forward and reverse primers are then selected based on knowledge of the full sequence of the nucleic acid sample of interest so as not to be capable of annealing to any part of the template other than their respective primer binding sequences during the nucleic acid amplification reaction.

In one embodiment both the forward and reverse amplification primers comprise a template-specific portion which is a sequence of at least 26 consecutive nucleotides.

In one embodiment the template-specific portion in the forward and/or reverse amplification primers is a sequence of at least 30 consecutive nucleotides.

In one embodiment the template-specific portion in the forward and/or reverse amplification primers is a sequence of from 30 to 35 consecutive nucleotides.

In one embodiment the template-specific portion in the forward and/or reverse amplification primers is a sequence of at least 35 consecutive nucleotides.

Preferably the template-specific portion in the forward and/or reverse amplification primers is a sequence of less than 50 consecutive nucleotides.

In a further embodiment the template-specific portion in the forward and/or reverse amplification primers is a sequence of from 30 to 45 consecutive nucleotides.

In a still further embodiment the template-specific portion in the forward and/or reverse amplification primers is a sequence of from 35 to 40 consecutive nucleotides.

In a still further embodiment the template-specific portion in the forward and/or reverse amplification primers is a sequence of 35 consecutive nucleotides.

In a second aspect the invention provides a method of nucleic acid sequencing which comprises amplifying one or more nucleic acid templates using a method according to the first aspect of the invention and carrying out a sequencing reaction to determining the sequence of the whole or a part of at least one amplified nucleic acid strand produced in the amplification reaction.

### BRIEF DESCRIPTION OF THE DRAWINGS

FIG. 1 illustrates a comparison of the results of cluster formation by nucleic acid amplification with different combinations of “long” amplification primers and standard length amplification primers. FIG. 1a. shows representative fluorescence CCD micrographs of nucleic acid colonies formed by amplification with different primer combinations following SyBr green staining. FIG. 1b. graphically illustrates the different fluorescence intensities achieved with different primer combinations.

FIG. 2 illustrates a comparison of the results of cluster formation by nucleic acid amplification with “long” amplification primers and standard length amplification primers using three different amplification templates. FIG. 2a. shows representative fluorescence CCD micrographs of nucleic acid colonies formed by amplification with different primer combinations following SyBr green staining. FIG. 1b. graphically illustrates the different fluorescence intensities achieved with different primer/template combinations.

### DETAILED DESCRIPTION OF THE INVENTION

The invention relates to a method of solid-phase nucleic acid amplification using forward and reverse amplification primers to amplify one or more template nucleic acids, which is characterised in that the forward and/or the reverse amplification primers comprise a template-specific portion which is a sequence of at least 26 consecutive nucleotides capable of annealing to a primer binding sequence in the template or the complement thereof and that the forward and reverse primers are not capable of annealing to any part of the template other than their respective primer binding sequences during the nucleic acid amplification reaction.

The term “solid-phase amplification” as used herein refers to any nucleic acid amplification reaction carried out on or in association with a solid support such that all or a portion of the amplified products are immobilised on the solid support as they are formed. In particularly, the term encompasses solid-phase polymerase chain reaction (solid-phase PCR), which is a reaction analogous to standard solution phase PCR, except that one or both of the forward and reverse amplification primers is/are immobilised on the solid support.

In order to carry out the amplification method of the invention the following components are brought into contact under conditions which permit a nucleic acid amplification reaction (e.g. PCR) to take place:

i) a solid support;
ii) forward and reverse amplification primers, with the proviso that the solid support must be provided with one or both of the forward and reverse amplification primers immobilised thereon. In a preferred embodiment the support will be provided with both forward and reverse primers already immobilised thereon; and
iii) one or more templates to be amplified with the forward and reverse primers, each template comprising at the 3′ end a sequence of nucleotides capable of annealing to the forward amplification primers and at the 5′ end a sequence of nucleotides the complement of which is capable of annealing to the reverse amplification primers.

By “conditions which permit a nucleic acid amplification reaction” is meant that the specified components must be brought together in a final reaction mixture in the presence of all the appropriate substrates (e.g. dNTPs), enzymes (e.g. Taq polymerase) buffer components etc required for the nucleic acid amplification reaction (e.g. PCR) and under the conditions of temperature (e.g. thermal cycling) required for the reaction to take place. Conditions for solid-phase amplification will be generally known to one skilled in the art. There are various ways in which the specified components may be brought together, as will be further described herein.

An essential difference between the method of the invention and prior art methods of solid-phase amplification lies in structure of the amplification primers. All PCR reactions, whether carried out in solution phase or on a solid support, require at least two amplification primers, often denoted “forward” and “reverse” primers, that are capable of annealing specifically to the template to be amplified under the conditions encountered in the “primer annealing step” of each cycle of the PCR reaction, although in certain embodiments the forward and reverse primers may be identical. Thus, all PCR primers must include a “template-specific portion”, the being a sequence of nucleotides capable of annealing to a primer-binding sequence in the template to be amplified (or the complement thereof if the template is viewed as a single strand) during the annealing step.

In the context of this application the term “template to be amplified” refers to the original or starting template added to the amplification reaction. The “template-specific portion” in the forward and reverse amplification primers refers to a sequence capable of annealing to the original or starting template present at the start of the amplification reaction and references to the length of the “template-specific portion” relate to the length of the sequence in the primer which anneals to the starting template. It will be appreciated that if the primers contain any nucleotide sequence which does not anneal to the starting template in the first amplification cycle then this sequence may be copied into the amplified products (assuming the primer does not contain any moiety which prevents read-through of the polymerase). Hence, the amplified strands produced in the first and subsequent cycles of amplification, which may serve as “templates” in subsequent amplification cycles, may be longer that the starting template. Such amplified strands are not intended to be encompassed by the term “template to be amplified”.

The present inventors have observed that the efficiency of solid-phase PCR amplification can be improved, whilst retaining specificity, by increasing the length of the template-specific portion of the forward and/or reverse amplification primers, beyond the standard length recommended in the prior art (typically 20-25 nucleotides).

Thus, the invention relates to the use of forward and reverse primers including a template-specific portion of at least 26 consecutive nucleotides.

In any given amplification reaction it is preferred for both the forward and reverse primers to include a template-specific portion of at least 26 consecutive nucleotides, although it is not essentially for the template-specific portions of the forward and reverse primers to be exactly the same length.

In one embodiment, the template-specific portion in the forward and/or reverse amplification primers may be a sequence of 30 or more consecutive nucleotides.

In one embodiment, the template-specific portion in the forward and/or reverse amplification primers may be a sequence of from 30 to 35 consecutive nucleotides.

In one embodiment, the template-specific portion in the forward and/or reverse amplification primers may be a sequence of 35 or more consecutive nucleotides.

In order to minimise the occurrence of mis-matching and non-specific priming it is generally be preferred that the template-specific portions of the forward and reverse amplification primers be no greater than 50 consecutive nucleotides in length.

In a further embodiment the template-specific portion in the forward and/or reverse amplification primers may be a sequence of from 30 to 45 consecutive nucleotides.

In a still further embodiment the template-specific portion in the forward and/or reverse amplification primers may be a sequence of from 35 to 40 consecutive nucleotides.

The nucleotide sequences of the template-specific portions of the forward and reverse primers are selected to achieve specific hybridisation to the template to be amplified under the conditions of the annealing steps of the amplification reaction, whilst minimising non-specific hybridisation to any other sequences present in the template. Skilled readers will appreciate that is it not strictly required for the template-specific portion to be 100% complementary to the template, a satisfactory level of specific annealing can be achieved with less than perfectly complementary sequences. In particular, one or two mis-matches in the template-specific portion can usually be tolerated without adversely affecting specificity for the template. Therefore, the term “template-specific portion” should not be interpreted as requiring 100% complementarity with the template. However, the requirement that the primers do not anneal non-specifically to regions of the template other than their respective primer-binding sequences must be fulfilled.

Amplification primers are generally single-stranded polynucleotide structures. They may contain a mixture of natural and non-natural bases and also natural and non-natural backbone linkages, provided that any non-natural modifications do not preclude function as a “primer”, that being defined as the ability to anneal to a template polynucleotide strand during the conditions of the amplification reaction and act as an initiation point for synthesis of a new polynucleotide strand complementary to the template strand.

Primers may additionally comprise non-nucleotide chemical modifications, again provided that such modifications do not prevent “primer” function. Chemical modifications may, for example, facilitate covalent attachment of the primer to a solid support. Certain chemical modifications may themselves improve the function of the molecule as a primer, or may provide some other useful functionality, such as for example providing a site for cleavage to enable the primer (or an extended polynucleotide strand derived therefrom) to be cleaved from the solid support.

As outlined above, the solid support may be provided with either one or both of the forward and reverse amplification primers already immobilised thereon. In certain embodiments, which will be further described hereinbelow, the solid-support may be provided with one or more templates to be amplified immobilised thereon in addition to the amplification primers. The general features of the solid support will also be described elsewhere herein.

Although the invention encompasses “solid-phase” amplification methods in which only one amplification primer is immobilised (the other primer usually being present in free solution), it is preferred for the solid support to be provided with both the forward and the reverse primers immobilised. In practice, there will be a “plurality” of identical forward primers and/or a “plurality” of identical reverse primers immobilised on the solid support, since the PCR process generally requires an excess of primers to sustain amplification. References herein to forward and reverse primers are to be interpreted accordingly as encompassing a “plurality” of such primers unless the context indicates otherwise.

As will be appreciated by the skilled reader, any given PCR reaction requires at least one type of forward primer and at least one type of reverse primer specific for the template to be amplified. However, in certain embodiments the forward and reverse primers may comprise template-specific portions of identical sequence, and may have entirely identical nucleotide sequence and structure (including any non-nucleotide modifications). In other words, it is possible to carry out the method of the invention using only one type of primer, provided that the essential features of the invention with respect to length of the template-specific portion etc are present. Other embodiments may use forward and reverse primers which contain identical template-specific sequences but which differ in some other structural features. For example one type of primer may contain a non-nucleotide modification which is not present in the other.

In other embodiments of the invention the forward and reverse primers may contain template-specific portions of different sequence.

In certain embodiments, two types of forward primers differing in some property may be used in conjunction with a single reverse primer (or vice versa). It is also possible to carry out “multiplex” PCR, in which two or more sets of forward and reverse primers are used to amplify two or more templates in parallel in a single reaction. All of these variations of the basic PCR reaction are contemplated by the invention in the context of “solid-phase” amplification.

When referring to immobilisation or attachment of molecules (e.g. nucleic acids such primers, templates etc.) to a solid support, the terms “immobilised” and “attached” are used interchangeably herein and both terms are intended to encompass direct or indirect, covalent or non-covalent attachment, unless indicated otherwise, either explicitly or by context. In certain embodiments of the invention covalent attachment may be preferred, but generally all that is required is that the molecules (e.g. nucleic acids) remain immobilised or attached to the support under the conditions in which it is intended to use the support, for example in applications requiring nucleic acid amplification and/or sequencing.

Certain embodiments of the invention make use of solid supports comprised of an inert substrate or matrix (e.g. glass slides, polymer beads etc) which is been “functionalised”, for example by application of a layer or coating of an intermediate material comprising reactive groups which permit covalent attachment to biomolecules, such as polynucleotides. Examples of such supports include, but are not limited to, polyacrylamide hydrogels supported on an inert substrate such as glass. In such embodiments, the biomolecules (e.g. polynucleotides) may be directly covalently attached to the intermediate material (e.g. the hydrogel) but the intermediate material may itself be non-covalently attached to the substrate or matrix (e.g. the glass substrate). The term “covalent attachment to a solid support” is to be interpreted accordingly as encompassing this type of arrangement.

In all embodiments of the invention, amplification primers are preferably immobilised by covalent attachment to the solid support at or near the 5′ end of the primer, leaving the template-specific portion of the primer free for annealing to it's cognate template and the 3′ hydroxyl group free for primer extension. Any suitable covalent attachment means known in the art may be used for this purpose. The chosen attachment chemistry will depend on the nature of the solid support, and any derivatisation or functionalisation applied to it. The primer itself may include a moiety, which may be a non-nucleotide chemical modification, to facilitate attachment. In one particularly preferred embodiment the primer may include a sulphur-containing nucleophile, such as phosphorothioate or thiophosphate, at the 5′ end. In the case of solid-supported polyacrylamide hydrogels (as described below), this nucleophile will bind to a “C” group present in the hydrogel. The most preferred means of attaching primers and templates to a solid support is via 5′ phosphorothioate attachment to a hydrogel comprised of polymerised acrylamide and N-(5-bromoacetamidylpentyl) acrylamide (BRAPA).

In a preferred embodiment of the method the forward and/or reverse amplification primers may include a linker portion, in addition to the template-specific portion. The term “linker portion” refers to a portion of the primer molecule positioned upstream of the 5′ end of the template-specific portion which is not capable of annealing to the template, or the complement thereof, under conditions used for the amplification reaction. Generally the “linker” portion, if present, occurs between the site if attachment to the solid support and the 5′ end of the template-specific portion of the primer, given the general structure: 5′-A-L-S-3′

wherein A represents a moiety which allows attachment to a solid support, L represents the optional linker portion and S is the template-specific portion. Moiety A may form a part of a larger linker moiety, the two elements do not have to be separable.

The linker portion may be comprised of natural or non-natural nucleotides, non-nucleotide chemical moieties, or any combination thereof.

The linker may be a carbon-containing chain such as those of formula (CH₂)ₙ wherein “n” is from 1 to about 1500, for example less than about 1000, preferably less than 100, e.g. from 2-50, particularly 5-25. However, a variety of other linkers may be employed with the only restriction placed on their structures being that the linkers are stable under conditions under which the primers are intended to be used, e.g. conditions used in DNA amplification and subsequent analysis of the amplification products (e.g. nucleic acid sequencing).

Linkers which do not consist of only carbon atoms may also be used. Such linkers include polyethylene glycol (PEG) having a general formula of (CH₂—CH₂—O)ₘ, wherein m is from about 1 to 600, preferably less than about 500, more preferably less than about 100.

Linkers formed primarily from chains of carbon atoms and from PEG may be modified so as to contain functional groups which interrupt the chains. Examples of such groups include ketones, esters, amines, amides, ethers, thioethers, sulfoxides, sulfones. Separately or in combination with the presence of such functional groups may be employed alkene, alkyne, aromatic or heteroaromatic moieties, or cyclic aliphatic moieties (e.g. cyclohexyl). Cyclohexyl or phenyl rings may, for example, be connected to a PEG or (CH₂)ₙ chain through their 1- and 4-positions.

As an alternative to the linkers described above, which are primarily based on linear chains of saturated carbon atoms, optionally interrupted with unsaturated carbon atoms or heteroatoms, other linkers may be envisaged which are based on nucleic acids or monosaccharide units (e.g. dextrose). It is also within the scope of this invention to utilise peptides as linkers.

A variety of non-nucleotide linker or spacer units suitable for use in primers according to the invention are commercially available from suppliers of reagents for automated chemical synthesis of oligonucleotides. By way of example, Fidelity Systems Inc., Gaithersburg, Md., USA supply a number of linker units based on phosphoramidite chemistry that can be incorporated into an otherwise polynucleotide chain using standard techniques and equipment for automated DNA synthesis.

Non-nucleotide chemical spacers will preferably be at least 20 atoms, and more preferably at least 40 atoms in length.

In a further embodiment the linker may comprise one or more nucleotides, preferably deoxyribonucleotides although ribonucleotide linkers and mixtures of deoxyribo- and ribonucleotides are not excluded. Such nucleotides may also be referred to herein as “linker” nucleotides. Typically from 1 to 20, more preferably from 1 to 15 or from 1 to 10, and more particularly 2, 3, 4, 5, 6, 7, 8, 9 or 10 linker nucleotides may be included. Most preferably the primer will include 10 linker nucleotides. It is preferred to use polyT spacers, although other nucleotides and combinations thereof can be used. In one preferred embodiment the primer may include 10T linker nucleotides.

Polynucleotide linkers are selected such that they are not capable of annealing to the starting template for the amplification (PCR) reaction in the first reaction cycle. It will be appreciated that a nucleotide-based linker will usually be copied during the amplification reaction, unless the primer contains any moiety which prevents read-through of the polymerase into the nucleotide-based linker portion of the primer. Thus, the amplified strands formed in the amplification reaction, which may serve as “templates” in further rounds of amplification, may include sequences derived from copying of the nucleotide-based linker portion. The linker portion of the primer will be capable of annealing to these amplified strands which contain a complementary sequence in subsequent cycles of amplification, even if it can not anneal to the original (starting) template in the first cycle. However, it is to be understood that references to the length of the “template-specific portion” in the forward and reverse amplification primers relate only to the length of the sequence which anneals to the starting template for amplification. Linker sequences which are capable of annealing to amplified strands but not to the starting template are not to be taken into account when determining the length of the “template-specific portion” in a given primer.

The template(s) for solid-phase amplification must include (when viewed as a single strand) at the 3′ end a “primer-binding sequence” which is a sequence of nucleotides capable of annealing to the forward amplification primer and at the 5′ end a “primer-binding sequence” which is a sequence of nucleotides the complement of which is capable of annealing to the reverse amplification primers. It will be appreciated, however, that the template to be amplified will commonly be in double-stranded form, in which case the complementary strand includes a sequence at the 3′ end a primer-binding sequence capable of annealing to the reverse amplification primers and at the 5′ end a primer-binding sequence the complement of which is capable of annealing to the forward amplification primers.

In this context the term “annealing” is to be given the same meaning as when used to refer to annealing between the template-specific portion of the primers and the template, i.e. it refers to specific hybridisation under the conditions used for the annealing steps of the amplification reaction.

The sequences in the template which permit annealing to the forward and reverse amplification primers are referred to herein as “primer binding sequences”. It will again be appreciated that 100% complementarity between the primer binding sequences and the template-specific portions of the primers is not absolutely required, although it is generally preferred.

Typically the templates to be amplified also include a “target sequence” which it is desired to amplify, the target sequence being located between the two primer binding sequences. The primer binding sequences may flank the target sequence such that they directly abut the target sequence, or further sequences of one or more nucleotides may be inserted between one or both of the primer binding sequences and the target sequence. For example, in certain embodiments a nucleotide sequence providing a binding site for a universal sequencing primer may be inserted between one of the primer binding sequences and the target sequence.

In certain embodiments, such as for example the amplification of genomic DNA fragments of a cDNA library, the target sequence may represent a fragment of the full sequence of a nucleic acid sample of interest (e.g. genomic DNA or a collection of cDNAs). In this context the fragment may typically be at least 300 bp, preferably at least 500 bp, typically in the range of from 300 bp to 1.5 kb or from 500 bp to 1 kb in length. Where the method is used to simultaneously amplify a plurality of template molecules, it is preferred that at least 90%, and preferably substantially all, of the templates include different target sequences.

The method of the invention may be used to amplify a single template or a mixture of templates which have different nucleotide sequences over all or a part of their length. In one embodiment the template may be a plurality or library of nucleic acid molecules which share common or “universal” primer binding sequences at their 5′ or 3′ ends flanking different target sequences to be amplified. Such a library of templates may be amplified using a pair of common or “universal” forward and reverse primers which incorporate template-specific sequences capable of annealing to the “universal” primer binding sequences. It is possible to use a single type of universal primer, or a universal primer-pair, in which forward and reverse primers contain template-specific portions of different sequence.

The precise nucleotide sequences of the template-specific portions of the forward and reverse primers are not particularly limited. However, it is a feature of the invention that the template-specific portions of the forward and reverse primers are sequences which are capable of annealing specifically to the primer-binding sequences in the template, but which exhibit minimal non-specific hybridisation with other sequences in the template under the conditions used in the annealing steps of the amplification reaction. Thus, the amplification primers do not anneal to regions of the template other than their respective primer-binding sequences during the amplification reaction.

The conditions encountered during the annealing steps of a solid-phase PCR reaction will be generally known to one skilled in the art, although the precise annealing conditions will vary from reaction to reaction. Typically such conditions may comprise, but are not limited to, (following a denaturing step at a temperature of about 94° C. for about one minute) exposure to a temperature in the range of from 50° C. to 65° C. (preferably 55-58° C.) for a period of about 1 minute in standard PCR reaction buffer, (optionally supplemented with 1M betain and 1.3% DMSO).

Suitable templates, or libraries of templates, to be amplified with universal primers may be prepared by modifying one or more target polynucleotides (embodying target sequences) that it is desired to amplify by addition of known adaptor sequences to the 5′ and 3′ ends. The target molecules themselves may be any polynucleotide molecules it is desired to amplify, of known, unknown or partially known sequence (e.g. random fragments of human genomic DNA). The adaptor sequences enable amplification of these molecules on a solid support to form clusters using forward and reverse primers incorporating universal template-specific sequences.

The adaptors are typically short oligonucleotides that may be synthesised by conventional means. The adaptors may be attached to the 5′ and 3′ ends of target nucleic acid fragments by a variety of means (e.g. subcloning, ligation. etc). More specifically, two different adaptor sequences are attached to a target nucleic acid molecule to be amplified such that one adaptor is attached at one end of the target nucleic acid molecule and another adaptor is attached at the other end of the target nucleic acid molecule. The target polynucleotides may advantageously be size-fractionated prior to modification with the adaptor sequences.

In embodiments of the invention which use forward and reverse primers including template-specific portions of identical sequence, it is possible to modify double-stranded target nucleic acid molecules by the addition of identical double-stranded adaptors to both ends of the target nucleic acid. The adaptors may be partially double-stranded provided that the primer-binding sequence is added in double-stranded form. Thus, when the individual strands of the double-stranded templates are denatured the single strands will contain the correct combination of primer-binding sequences required for the amplification reaction.

In embodiments of the method which use “universal” forward and reverse primers incorporating template-specific sequences capable of annealing specifically to respective “universal” primer binding sequences in the template, the “universal” primers (and particularly the template-specific portions thereof) should not anneal to any other region of the template during the amplification reaction.

In a preferred embodiment of the invention, the templates to be amplified may comprise a library or collection of genomic DNA fragments flanked by universal primer-binding sequences. The fragments will typically be at least 300 bp, preferably at least 500 bp, typically in the range of from 300 bp to 1.5 kb or from 500 bp to 1 kb in length. Most preferably the genomic DNA fragments will be fragments of human genomic DNA. The templates for a single amplification reaction may be derived from a library fragments which represent a whole genome (e.g. a whole human genome) or a part of a genome, with each individual template comprising one fragment from the library flanked by appropriate primer binding sequences. The “part” of a genome will typically comprise more than one single gene and may comprise, for example, from 50 to 100% of the complete genome, a single chromosome or any combination of two or more chromosomes. The method may be applied to a plurality of target molecules derived from a common source, for example a library of genomic DNA fragments derived from one individual or pooled samples from several individuals. Techniques for fragmentation of genomic DNA include, for example, enzymatic digestion or mechanical shearing.

The method of the invention can also be applied to the amplification of target fragments derived from other complex mixtures of nucleic acids, such as for example collections of cDNAs or fragments thereof.

In embodiments of the method where it is intended to amplify target sequences which represent fragments of a nucleic acid of interest (e.g. genomic DNA fragments), the forward and reverse primers are selected such that they do not bind to any sequence within the full sequence of the nucleic acid of interest (e.g. the genomic DNA from which the fragments included in the templates to be used for that particular amplification reaction were derived). Where the templates include genomic sequences, and more specifically human genomic sequences, the template-specific sequences in the forward and reverse amplification primers should ideally be selected such that any sequence of 20 consecutive nucleotides in the template-specific sequence is at least 2 bases different to any 20-mer in either strand of the respective genome (e.g. the human genome).

A problem is encountered when designing primers with “long” template-specific sequences, i.e. at least 26 nucleotides, in eliminating or minimising non-specific annealing to regions of the template other than the intended primer-binding sequences. Generally, as the length of the template-specific portion increases, so too does the possibility for non-specific annealing. Thus, it is more difficult to satisfy the requirement for no or minimal non-specific annealing to the template as the length of the template-specific portion is increased.

The inventors have tackled this problem by adopting an approach to primer design in which candidate sequences shorter that the intended length of the template-specific sequence are compared with the whole genome sequence and then assembled into a template-specific sequence of the desired length. This method can be used to select suitable primer sequences for use in amplifying target sequences which represent fragments of the full sequence of a nucleic acid of interest based on knowledge of the full sequence of the nucleic acid of interest.

By way of example, “long” template-specific sequences which exhibit minimal non-specific annealing to genomic sequences can be designed by the following approach. To begin with a large number (e.g. 500000) of “short” sequences (e.g. 15-mers for the human genome) are randomly chosen. The short sequences will be shorter than the intended length of the template-specific portion and should ideally be just long enough to represent a unique sequence in the genome of interest. The random short sequences are then matched to the chosen genome sequence (e.g. the human genome) using an algorithm that finds all matches on either strand of the genome with 2 errors or less. Short sequences which have at least 2 differences between any 15-mer on either strand of the genome are selected and any that match with 2 differences or less to another selected short sequence are excluded. The remaining short sequences are chosen as “seeds”.

The seed short sequences are then paired (e.g. to make 30-mers). Pairing is generally done as follows.

i) Pick the seed sequence X having the least number of 2-difference matches to the genome.

ii) Pick the seed sequence Y having the next-least number of 2-difference matches to the genome.

iii) Pair them up into a 30-mer and check for:
A. No 3×2 repeats e.g. GCGCGC
B. No 2×3 repeats e.g. GACGAC
C. No 3×1 repeats e.g. GGG
D. Does not start with GG or CC

If none of A to D apply, then X and Y are a successful pair, thus they are paired up and excluded from further pairing. If one or more of A to D is found, step ii) is repeated with an alternative Y seed sequence. If step ii) is repeated until there are no longer any potential Y sequences left to pair with X, then X is excluded from further pairing.

When a successful pair is identified, the middle 20-mer of the pair is matched to the genome. Any that match the genome with 2 errors or less are rejected. Remaining pairs (e.g. 30 mers) can be used as the basis of a template-specific sequence. Appropriate modifications, such as a linker portion, may be added to the template-specific sequence in the final primer.

In order to create longer template-specific sequences, additional bases may be added to the “paired” seed sequences identified using the process described above (e.g. 5 bases to each 30-mer to create a set of 35-mers). The additional sequences must be selected such that the last 20 bases of each are at least 2 bases different from any 20-mer on either strand of the genome. This can be done by checking the results for all possible additional sequences (e.g. 5-mers) tacked on to each paired sequence (e.g. 30 mers). The additional sequences should not violate conditions A to D above.

Lastly, the last 15 bases of each extended sequence (e.g. 35-mer) should be checked to ensure that it is at least 2 bases different from:

i) the last 15 bases of any other extended sequence (35-mer)
ii) the first 15 bases of any other extended sequence (35-mer)
iii) bases 15-30 of any other extended sequence (35-mer)

The resulting extended sequences may then be assessed for secondary structure using standard predictive software for oligonucleotide design. Changes may be made in silico to these sequences to remove potential secondary structure and the resulting primer sequences reanalysed by taking the first 20 bases, the last 20 bases and the middle 20 bases of each primer and matching these 20-mers to both strands of the genome. All of these 20-mers should be at least 2 bases different to any 20-mer in the genome.

Skilled readers will appreciate that this protocol may be varied in order to create template-specific sequences of different lengths, for example by varying that length of the short “seed” sequences, and/or by varying the number of additional nucleotides. Shorter sequences could even be derived by removing nucleotides from one or both ends of the “paired” seed sequences.

Suitable “universal” primer-pairs for use in the method of the invention include, but are not limited to, the following:

(SEQ ID NO:1)

A) 5′-PS-linker-gctggcacgtccgaacgcttcgttaatccgttga
g-3′
In combination with any one of

(SEQ ID NO:2)

B) 5′-PS-linker-cgtcgtctgccatggcgcttcggtggatatgaac
t-3′

(SEQ ID NO:3)

C) 5′-PS-linker-acggccgctaatatcaacgcgtcgaatccgcaac
t-3′

(SEQ ID NO:4)

D) 5′-PS-linker-gccgcgttacgttagccggactattcgatgcag
c-3′
or

(SEQ ID NO:5)

5′-PS-linker-cgaattcactagtgattaatgatacggcgaccaccg
a-3′
in combination with

(SEQ ID NO:6)

5′-PS-linker-gcgggaattcgattcaagcagaagacggcatacg
a-3′

wherein PS represents a phosphorothioate moiety and -linker- is preferably a 10T polynucleotide linker or a PEG-based linker.

Primers B and C can each also be used alone in a single primer amplification reaction.

In certain embodiments of the invention the template(s) to be amplified may be immobilised on the solid support, preferably via covalent attachment at the 5′ end. Thus, in part a) of the method the solid support may be provided with the template(s) already covalently attached thereto, in addition to the forward and/or the reverse amplification primers. In order to enable covalent attachment the template(s) may be modified at or near the 5′ end. The means of attachment of the template(s) to the solid support may conveniently be the same means used for attachment of the amplification primers. Hence, any preferred means described herein in the context of primer attachment may also be used, and are indeed preferred, for template attachment. In one particularly preferred embodiment the template(s) may include a 5′ thiophosphate or phosphorothioate group to facilitate covalent attachment to a surface.

It is most preferred to use the method of the invention to form clustered arrays of nucleic acid colonies, analogous to those described in WO 00/18957 and WO 98/44151. The terms “cluster” and “colony” are used interchangeably herein to refer to a discrete site on a solid support comprised of a plurality of identical immobilised nucleic acid strands and a plurality of identical immobilised complementary nucleic acid strands. The term “clustered array” refers to an array formed from such clusters or colonies. In this context the term “array” is not to be understood as requiring an ordered arrangement of clusters. Clustered arrays are generally formed by solid-phase PCR amplification.

As aforesaid, there are various ways in which the components specified in part a) of the method of the invention may be brought together for a solid-phase amplification reaction.

In one embodiment, analogous to the amplification method described in WO 00/18957, both forward and reverse amplification primers and the template to be amplified are immobilised to the solid support at the 5′ end prior (e.g. by covalent attachment) to solid-phase amplification. The process of attachment of primers (and templates) to the solid support may be referred to herein as “grafting”. In this embodiment of the method, the forward and reverse primers and the templates to be amplified may be mixed together in solution and then grafted onto the solid support in a single grafting step. Amplification may then proceed substantially as described in WO 00/18957.

In a further embodiment, analogous to the amplification method described in WO 98/44151, the forward and reverse primers are first grafted onto the solid support in an initial step, and then denatured template strands are annealed to the immobilised primers. Amplification may then proceed substantially as described in WO 98/44151, the first step of the amplification reaction being a primer extension step.

The term “solid support”, as used herein, refers to the material to which the polynucleotides molecules are attached. Suitable solid supports are available commercially, and will be apparent to the skilled person. The supports can be manufactured from materials such as glass, ceramics, silica and silicon. Supports with a gold surface may also be used. The supports usually comprise a flat (planar) surface, or at least a structure in which the polynucleotides to be interrogated are in approximately the same plane. In other embodiments the solid support may be non-planar, or may even be formed from a plurality of discrete units, e.g. microbeads. Supports of any suitable size may be used. For example, planar supports might be on the order of 1-10 cm in each direction.

Preferred supports include, but are not limited to, solid-supported polyacrylamide hydrogels.

In preparing hydrogel-based solid-supported molecular arrays, a hydrogel is formed and molecules displayed from it. These two features—formation of the hydrogel and construction of the array—may be effected sequentially or simultaneously.

Where the hydrogel is formed prior to formation of the array, it is typically produced by allowing a mixture of comonomers to polymerise. Generally, the mixture of comonomers contain acrylamide and one or more comonomers, the latter of which permit, in part, subsequent immobilisation of molecules of interest so as to form the molecular array.

The comonomers used to create the hydrogel typically contain a functionality that serves to participate in crosslinking of the hydrogel and/or immobilise the hydrogel to the solid support and facilitate association with the target molecules of interest.

Generally, as is known in the art, polyacrylamide hydrogels are produced as thin sheets upon polymerisation of aqueous solutions of acrylamide solution. A multiply unsaturated (polyunsaturated) crosslinking agent (such as bisacrylamide) is generally present; the ratio of acrylamide to bisacrylamide is generally about 19:1. Such casting methods are well known in the art (see for example Sambrook et al., 2001, Molecular Cloning, A Laboratory Manual, 3rd Ed, Cold Spring Harbor Laboratory Press, Cold Spring Harbor Laboratory Press, NY) and need not be discussed in detail here.

Some form of covalent surface modification of the solid support may be practised in order to achieve satisfactory immobilisation of either hydrogel-based molecular arrays or hydrogels to which it is desired to array molecules. However, it has been observed that such functional modification of the support is not necessary in order to achieve satisfactory immobilisation of arrays of polynucleotides. In order to make useful supported arrays capable of binding molecules of interest, a mixture of comonomers comprising at least one hydrophilic monomer and a functionalised comonomer (functionalised to the extent that the monomer once incorporated into the polymer is capable of binding the molecule of interest to the surface of the hydrogel) may be polymerised so as to form a hydrogel capable of being immobilised on a solid supported, preferably a silica-based, substrate. In particular, the hydrogel may be substantially free of any binder silane components.

In one embodiment the hydrogel may be formed by a method comprising polymerising on said support a mixture of:

(i) a first comonomer which is acrylamide, methacrylamide, hydroxyethyl methacrylate or N-vinyl pyrrolidinone; and

(ii) a second comonomer which is a functionalised acrylamide or acrylate of formula (I):

H₂C═C(H)—C(═O)-A-B-C (I);

or a methacrylate or methacrylamide of formula (II):

or H₂C═C(CH₃)—C(═O)-A-B-C— (II)

(wherein:

A is NR or O, wherein R is hydrogen or an optionally substituted saturated hydrocarbyl group comprising 1 to 5 carbon atoms;

-B- is an optionally substituted alkylene biradical of formula —(CH₂)ₙ— wherein n is an integer from 1 to 50; and wherein n=2 or more, one or more optionally substituted ethylene biradicals —CH₂CH₂— of said alkylene biradical may be independently replaced by ethenylene and ethynylene moieties; and wherein n=1 or more, one or more methylene biradicals —CH₂— may be replaced independently with an optionally substituted mono- or polycyclic hydrocarbon biradical comprising from 4 to 50 carbon atoms, or a corresponding heteromonocyclic or heteropolycyclic biradical wherein at least 1 CH₂ or CH₂ is substituted by an oxygen sulfur or nitrogen atom or an NH group; and

C is a group for reaction with a compound to bind said compound covalently to said hydrogel) to form a polymerised product,

characterised in that said method is conducted on, and immobilises the polymerised product to, said support which is not covalently surface-modified.

It has been found that omission of a covalent surface-modification step (particularly of the solid support) affords a surface having greater passivity than in the prior art, particularly when compared to those instances where the use of the silane-modifying agents described above with silica-based substrates are employed.

The solid upon which the hydrogel is supported is not limited to a particular matrix or substrate. Suitable supports include silica-based substrates, such as glass, fused silica and other silica-containing materials; they may also be silicone hydrides or plastic materials such as polyethylene, polystyrene, poly(vinyl chloride), polypropylene, nylons, polyesters, polycarbonates and poly(methyl methacrylate). Preferred plastics material are poly(methyl methacrylate), polystyrene and cyclic olefin polymer substrates. Alternatively, other solid supports may be used such as gold, titanium dioxide, or silicon supports. The foregoing lists are intended to be illustrative of, but not limited to, the invention. Preferably, the support is a silica-based material or plastics material such as discussed herein.

The methods by which the mixture of comonomers are polymerised in the invention are not characteristic of this invention and will be known to the skilled person (e.g. by recourse to Sambrook et al. (supra). Generally, however, the polymerisation will be conducted in an aqueous medium, and polymerisation initiated by any suitable initiator. Potassium or ammonium persulfate as an initiator is typically employed. Tetramethylethylenediamine (TMEDA or TEMED) may be and generally is used to accelerate the polymerisation.

It is not necessary that a polyunsaturated crosslinking agent such as bisacrylamide or pentaerythritol tetraacrylate is present in the mixture which is polymerised; nor is it necessary to form PRP-type intermediates and crosslink them.

Generally, in producing hydrogels according to this invention, only one compound of formulae (I) or (II) will be used. Use of a compound of the formulae (I) or (II) permits formation of a hydrogel capable of being immobilised to solid supports, preferably silica-based solid supports. The compounds of these formulae comprise portions A, B and C as defined herein.

Biradical A may be oxygen or N(R) wherein R is hydrogen or a C₁₋₅ alkyl group. Preferably, R is hydrogen or methyl, particularly hydrogen. Where R is a C₁₋₅ alkyl group, this may contain one or more, e.g. one to three substituents. Preferably, however, the alkyl group is unsubstituted.

Biradical B is a predominantly hydrophobic linking moiety, connecting A to C and may be an alkylene biradical of formula —(CH₂)ₙ—, wherein n is from 1 to 50. Preferably n is 2 or more, e.g. 3 or more. Preferably n is 2 to 25, particularly 2 to 15, more particularly 4 to 12, for example 5 to 10.

Where n in —(CH₂)ₙ— is 2 or more, one or more biradicals —CH₂CH₂— (-ethylene-) may be replaced with ethenylene or ethynylene biradicals. Preferably, however, the biradical B does not contain such unsaturation.

Additionally, or alternatively, where n in —(CH₂)ₙ— is 1 or more, one or more methylene radicals —CH₂— in B may be replaced with a mono- or polycyclic biradical which preferably comprises 5 to 10 carbon atoms e.g. 5 or 6 carbon atoms. Such cyclic biradicals may be, for example, 1,4-, 1,3- or 1,2-cyclohexyl biradicals. Bicylic radicals such as napthyl or decahydronaphthyl may also be employed. Corresponding heteroatom-substituted cyclic biradicals to those homocyclic biradicals may also be employed, for example pyridyl, piperidinyl, quinolinyl and decahydroquinolinyl.

It will be appreciated that the scope of -B- is thus not particularly restricted. Most preferably, however, -B- is a simple, unsubstituted, unsaturated alkylene biradical such as a C₃-C₁₀ alkylene group, optimally C₅-C₈, such as n-pentylene: —(CH₂)₅—.

Where an alkyl group (or alkylene, alkenylene etc) is indicated as being (optionally) substituted, substituents may be selected from the group comprising hydroxyl, halo (i.e. bromo, chloro, fluoro or iodo), carboxyl, aldehydro, amine and the like. The biradical -B- is preferably unsubstituted or substituted by fewer than 10, preferably fewer than 5, e.g. by 1, 2 or 3 such substituents.

Group C serves to permit attachment of molecules of interest after formation of the hydrogel. The nature of Group C is thus essentially unlimited provided that it contains a functionality allowing reaction between the hydrogel and the molecules to be immobilised. Preferably, such a functionality will not require modification prior to reaction with the molecule of interest and thus the C group is ready for direct reaction upon formation of the hydrogel. Preferably such a functionality is a hydroxyl, thiol, amine, acid (e.g. carboxylic acid), ester and haloacetamido, haloacetamido and in particular bromoacetamido being particularly preferred. Other appropriate C groups will be evident to those skilled in the art and include groups comprising a single carbon-carbon double bond which is either terminal (i.e. where a C group has an extremity terminating in a carbon-carbon double bond) or where the carbon-carbon double bond is not at a terminal extremity. When a C group comprises a carbon-carbon double bond, this is preferably fully substituted with C₁₋₅ alkyl groups, preferably methyl or ethyl groups, so that neither carbon atom of the C═C moiety bears a hydrogen atom.

The C moiety may thus comprise, for example, a dimethylmaleimide moiety as disclosed in U.S. Pat. No. 6,372,813, WO01/01143, WO02/12566 and WO03/014394.

The (meth)acrylamide or (meth)acrylate of formula (I) or (II) which is copolymerised with acrylamide, methacrylamide, hydroxyethyl methacrylate or N-vinyl pyrrolidinone is preferably an acrylamide or acrylate, i.e. of formula (I). More preferably it is an acrylamide and still more preferably it is an acrylamide in which A is NH.

The reaction between a comonomer of formula (I) or (II) and acrylamide, methacrylamide, hydroxyethyl methacrylate or N-vinyl pyrrolidinone methacrylamide, particularly acrylamide, has been found to afford hydrogels particularly suitable for use in the generation of molecular arrays. However, it will be appreciated by those skilled in the art that analogous copolymers may be formed by the reaction between comonomers of formula (I) or (II) and any vinylogous comonomer, hydroxyethylmethacrylate and n-vinyl pyrrolidinone being two examples of such vinylogous comonomers.

Control of the proportion of monomer of formula (I) or (II) to that of the first comonomer (e.g. acrylamide and/or methacrylamide, preferably acrylamide) allows adjustment of the physical properties of the hydrogel obtained on polymerisation. It is preferred that the comonomer of formula (I) or (II) is present in an amount of ≧1 mol %, preferably ≧2 mol % (relative to the total molar quantity of comonomers) in order for the hydrogel to have optimum thermal and chemical stability under conditions typically encountered during the preparation, and subsequent manipulation, of the molecular arrays produced from the hydrogels. Preferably, the amount of comonomer of formula (I) or (II) is less than or equal to about 5 mol %, more preferably less than or equal to about 4 mol %. Typical amounts of comonomer of formula (I) or (II) used are 1.5-3.5 mol %, exemplified herein by about 2% and about 3%.

The amounts of acrylamide or methacrylamide from which the hydrogels are primarily constructed are those typically used to form hydrogels, e.g. about 1 to about 10% w/v, preferably less than 5 or 6% w/v, e.g. about 1 to about 2% w/v. Again, of course, the precise nature of the hydrogel may be adjusted by, in part, control of the amount of acrylamide or methacrylamide used.

When forming the hydrogels, acrylamide or methacrylamide may be dissolved in water and mixed with a solution of a comonomer of formula (I) or (II). The latter may be conveniently dissolved in a water-miscible solvent, such as dimethylformamide (DMF), or water itself. The most appropriate solvent may be selected by the skilled person and shall depend upon the structure of the comonomer of formula (I) or (II).

The methods by which the monomers of formula (I) or (II) are synthesised will be evident to those skilled in the art. By way of example, the synthesis of a particularly preferred monomer (of formula (I) wherein A=NH, -B-=—(CH₂)₅— and —C═—N(H)—C(═O)CH₂Br is provided as an example hereinafter.

As noted above, the general methods by which the polymerisation is carried out are known to those skilled in the art. For example, generally acrylamide or methacrylamide is dissolved in purified water (e.g. Milli Q) and potassium or ammonium persulfate dissolved separately in purified water. The comonomer of formula (I) or (II) may be conveniently dissolved in a water-miscible organic solvent, e.g. glycerol, ethanol, methanol, dimethylformamide (DMF) etc. TEMED may be added as appropriate. Once formulated (a typical preparation is described in the examples), the mixture is polymerised with as little delay as possible after its formulation. The polymerisation process may be conducted by any convenient means.

### Use in Sequencing/Methods of Sequencing

The invention also encompasses methods of sequencing amplified nucleic acids generated using the methods of the invention. Thus, the invention provides a method of nucleic acid sequencing comprising amplifying one or more nucleic acid templates using a method according to the first aspect of the invention and carrying out a nucleic acid sequencing reaction to determine the sequence of the whole or a part of at least one amplified nucleic acid strand produced in the amplification reaction.

Sequencing can be carried out using any suitable “sequencing-by-synthesis” technique, wherein nucleotides are added successively to a free 3′ hydroxyl group, resulting in synthesis of a polynucleotide chain in the 5′ to 3′ direction. The nature of the nucleotide added is preferably determined after each addition.

The initiation point for the sequencing reaction may be provided by annealing of a sequencing primer to a product of the solid-phase amplification reaction.

The products of solid-phase amplification reactions wherein both forward and reverse amplification primers are covalently immobilised on the solid surface are so-called “bridged” structures formed by annealing of pairs of immobilised polynucleotide strands and immobilised complementary strands, both strands being attached to the solid support at the 5′ end. Arrays comprised of such bridged structures provide inefficient templates for nucleic acid sequencing, since hybridisation of a conventional sequencing primer to one of the immobilised strands is not favoured compared to annealing of this strand to its immobilised complementary strand under standard conditions for hybridisation.

In order to provide more suitable templates for nucleic acid sequencing it is preferred to remove substantially all or at least a portion of one of the immobilised strands in the “bridged” structure in order to generate a template which is at least partially single-stranded. The portion of the template which is single-stranded will thus be available for hybridisation to a sequencing primer. The process of removing all or a portion of one immobilised strand in a “bridged” double-stranded nucleic acid structure may be referred to herein as “linearisation”.

It will be appreciated that a linearization step may not be essential if the amplification reaction is performed with only one primer covalently immobilised and the other in free solution.

Bridged template structures may be linearised by cleavage of one or both strands with a restriction endonuclease or by cleavage of one strand with a nicking endonuclease. Other methods of cleavage can be used as an alternative to restriction enzymes or nicking enzymes. Preferred methods include the following:

### i) Chemical Cleavage

The term “chemical cleavage” encompasses any method which utilises a non-nucleic acid and non-enzymatic chemical reagent in order to promote/achieve cleavage of one or both strands of a double-stranded nucleic acid molecule. If required, one or both strands of the double-stranded nucleic acid molecule may include one or more non-nucleotide chemical moieties and/or non-natural nucleotides and/or non-natural backbone linkages in order to permit chemical cleavage reaction. In a preferred embodiment the modification(s) required to permit chemical cleavage may be incorporated into an amplification primer used in solid-phase nucleic acid amplification.

In a preferred but non-limiting embodiment one strand of the double-stranded nucleic acid molecule (or the amplification primer from which this strand is derived if formed by solid-phase amplification) may include a diol linkage which permits cleavage by treatment with periodate (e.g. sodium periodate). It will be appreciate that more than one diol can be included at the cleavage site.

Diol linker units based on phosphoramidite chemistry suitable for incorporation into polynucleotide chains are commercially available from Fidelity systems Inc. (Gaithersburg, Md., USA). One or more diol units may be incorporated into a polynucleotide using standard methods for automated chemical DNA synthesis. Hence, oligonucleotide primers including one or more diol linkers can be conveniently prepared by chemical synthesis.

In order to position the diol linker at an optimum distance from the solid support one or more spacer molecules may be included between the diol linker and the site of attachment to the solid support. The spacer molecule may be a non-nucleotide chemical moiety. Suitable spacer units based on phosphoramidite chemistry for use in conjunction with diol linkers are also supplied by Fidelity Systems Inc. One suitable spacer for use with diol linkers is the spacer denoted arm 26, identified in the accompanying examples. To enable attachment to a solid support at the 5′ end of the polynucleotide strand arm 26 may be modified to include a phosphorothioate group. The phosphorothioate group can easily be attached during chemical synthesis of a “polynucleotide” chain including the spacer and diol units.

Other spacer molecules could be used as an alternative to arm 26. For example, a stretch of non-target “spacer” nucleotides may be included. Typically from 1 to 20, more preferably from 1 to 15 or from 1 to 10, and more particularly 2, 3, 4, 5, 6, 7, 8, 9 or 10 spacer nucleotides may be included. Most preferably 10 spacer nucleotides will be positioned between the point of attachment to the solid support and the diol linker. It is preferred to use polyT spacers, although other nucleotides and combinations thereof can be used. In one preferred embodiment the primer may include 10T spacer nucleotides.

The diol linker is cleaved by treatment with a “cleaving agent”, which can be any substance which promotes cleavage of the diol. The preferred cleaving agent is periodate, preferably aqueous sodium periodate (NaIO₄). Following treatment with the cleaving agent (e.g. periodate) to cleave the diol, the cleaved product may be treated with a “capping agent” in order to neutralise reactive species generated in the cleavage reaction. Suitable capping agents for this purpose include amines, such as ethanolamine. Advantageously, the capping agent (e.g. ethanolamine) may be included in a mixture with the cleaving agent (e.g. periodate) so that reactive species are capped as soon as they are formed.

The combination of a diol linkage and cleaving agent (e.g. periodate) to achieve cleavage of one strand of a double-stranded nucleic acid molecule is preferred for linearisation of nucleic acid molecules on solid supported polyacrylamide hydrogels because treatment with periodate is compatible with nucleic acid integrity and with the chemistry of the hydrogel surface. However, utility of diol linkages/periodate as a method of linearisation is not limited to polyacrylamide hydrogel surfaces but also extends to linearisation of nucleic acids immobilised on other solid supports and surfaces, including supports coated with functionalised silanes (etc).

In a further embodiment, the strand to be cleaved (or the amplification primer from which this strand is derived if prepared by solid-phase amplification) may include a disulphide group which permits cleavage with a chemical reducing agent, e.g. Tris (2-carboxyethyl)-phosphate hydrochloride (TCEP).

### ii) Cleavage of Abasic Sites in a Double-Stranded Molecule

An “abasic site” is defined as a nucleoside position in a polynucleotide chain from which the base component has been removed. Abasic sites can occur naturally in DNA under physiological conditions by hydrolysis of nucleoside residues, but may also be formed chemically under artificial conditions or by the action of enzymes. Once formed, abasic sites may be cleaved (e.g. by treatment with an endonuclease or other single-stranded cleaving enzyme, exposure to heat or alkali), providing a means for site-specific cleavage of a polynucleotide strand.

In a preferred but non-limiting embodiment an abasic site may be created at a pre-determined position on one strand of a double-stranded polynucleotide and then cleaved by first incorporating deoxyuridine (U) at a pre-determined cleavage site in one strand of the double-stranded nucleic acid molecule. This can be achieved, for example, by including U in one of the primers used for preparation of the double-stranded nucleic acid molecule by solid-phase PCR amplification. The enzyme uracil DNA glycosylase (UDG) may then be used to remove the uracil base, generating an abasic site on one strand. The polynucleotide strand including the abasic site may then be cleaved at the abasic site by treatment with endonuclease (e.g EndoIV endonuclease, AP lyase, FPG glycosylase/AP lyase, EndoVIII glycosylase/AP lyase), heat or alkali.

Abasic sites may also be generated at non-natural/modified deoxyribonucleotides other than deoxyuridine and cleaved in an analogous manner by treatment with endonuclease, heat or alkali. For example, 8-oxo-guanine can be converted to an abasic site by exposure to FPG glycosylase. Deoxyinosine can be converted to an abasic site by exposure to AlkA glycosylase. The abasic sites thus generated may then be cleaved, typically by treatment with a suitable endonuclease (e.g. EndoIV, AP lyase). If the non-natural/modified nucleotide is to be incorporated into an amplification primer for use in solid-phase amplification, then the non-natural/modified nucleotide should be capable of being copied by the polymerase used for the amplification reaction.

In one embodiment, the molecules to be cleaved may be exposed to a mixture containing the appropriate glycosylase and one or more suitable endonucleases. In such mixtures the glycosylase and the endonuclease will typically be present in an activity ratio of at least about 2:1.

This method of cleavage has particular advantages in relation to the creation of templates for nucleic acid sequencing. In particular, cleavage at an abasic site generated by treatment with a glycosylase such as UDG generates a free 3′ hydroxyl group on the cleaved strand which can provide an initiation point for sequencing a region of the complementary strand. Moreover, if the starting double-stranded nucleic acid contains only one cleavable (e.g. uracil) base on one strand then a single “nick” can be generated at a unique position in this strand of the duplex. Since the cleavage reaction requires a residue, e.g. deoxyuridine, which does not occur naturally in DNA, but is otherwise independent of sequence context, if only one non-natural base is included there is no possibility of glycosylase-mediated cleavage occurring elsewhere at unwanted positions in the duplex. In contrast, were the double-stranded nucleic acid to be cleaved with a “nicking” endonuclease that recognises a specific sequence, there is a possibility that the enzyme may create nicks at “other” sites in the duplex (in addition to the desired cleavage site) if these possess the correct recognition sequence. This could present a problem if nicks are created in the strand it is intended to sequence rather than the strand that will be fully or partially removed to create the sequencing template and is a particular risk if the target portion of the double-stranded nucleic acid molecule is of unknown sequence.

The fact that there is no requirement for the non-natural (e.g. uracil) residue to be located in a detailed sequence context in order to provide a site for cleavage using this approach is itself advantageous. In particular, if the cleavage site is to be incorporated into an amplification primer to be used in the production of a clustered array by solid-phase amplification, it is necessarily only to replace one natural nucleotide (e.g. T) in the primer with a non-natural nucleotide (e.g. U) in order to enable cleavage. There is no need to engineer the primer to include a restriction enzyme recognition sequence of several nucleotides in length. Oligonucleotide primers including U nucleotides, and the other non-natural nucleotides listed above, can easily be prepared using conventional techniques and apparatus for chemical synthesis of oligonucleotides.

Another advantage gained by cleavage of abasic sites in a double-stranded molecule generated by action of UDG on uracil is that the first base incorporated in a “sequencing-by-synthesis” reaction initiating at the free 3′ hydroxyl group formed by cleavage at such a site will always be T. Hence, if the double-stranded nucleic acid molecule forms part of a clustered array comprised of many such molecules, all of which are cleaved in this manner to produce sequencing templates, then the first base universally incorporated across the whole array will be T. This can provide a sequence-independent assay for cluster intensity at the start of a sequencing “run”.

iii) Cleavage of Ribonucleotides

Incorporation of one or more ribonucleotides into a polynucleotide strand which is otherwise comprised of deoxyribonucleotides (with or without additional non-nucleotide chemical moieties, non-natural bases or non-natural backbone linkages) can provide a site for cleavage using a chemical agent capable of selectively cleaving the phosphodiester bond between a deoxyribonucleotide and a ribonucleotide or using a ribonuclease (RNAse). Therefore, sequencing templates can be produced by cleavage of one strand of a “bridged” structure at a site containing one or more consecutive ribonucleotides using such a chemical cleavage agent or an RNase. Preferably the strand to be cleaved contains a single ribonucleotide to provide a site for chemical cleavage.

Suitable chemical cleavage agents capable of selectively cleaving the phosphodiester bond between a deoxyribonucleotide and a ribonucleotide include metal ions, for example rare-earth metal ions (especially La particularly Tm³⁺, Yb³⁺ or Lu³⁺ (Chen et al. Biotechniques. 2002, 32: 518-520; Komiyama et al. Chem. Commun. 1999, 1443-1451)), Fe(3) or Cu(3), or exposure to elevated pH, e.g. treatment with a base such as sodium hydroxide. By “selective cleavage of the phosphodiester bond between a deoxyribonucleotide and a ribonucleotide” is meant that the chemical cleavage agent is not capable of cleaving the phosphodiester bond between two deoxyribonucleotides under the same conditions.

The base composition of the ribonucleotide(s) is generally not material, but can be selected in order to optimise chemical (or enzymatic) cleavage. By way of example, rUMP or rCMP are generally preferred if cleavage is to be carried out by exposure to metal ions, especially rare earth metal ions.

The ribonucleotide(s) will typically be incorporated into one strand of a “bridged” double-stranded nucleic acid molecule (or the amplification primer from which this strand is derived if prepared by solid-phase amplification), and may be situated in a region of the bridged structure which is single-stranded when the two complementary strands of the double-stranded molecule are annealed (i.e. in a 5′ overhanging portion). If the double-stranded nucleic acid molecule is prepared by solid-phase PCR amplification using forward and reverse amplification primers, one of which contains at least one ribonucleotide, the standard DNA polymerase enzymes used for PCR amplification are not capable of copying ribonucleotide templates. Hence, the PCR products will contain an overhanging 5′ region comprising the ribonucleotide(s) and any remainder of the amplification primer upstream of the ribonucleotide(s).

The phosphodiester bond between a ribonucleotide and a deoxyribonucleotide, or between two ribonucleotides, may also be cleaved by an RNase. Any endocytic ribonuclease of appropriate substrate specificity can be used for this purpose. If the ribonucleotide(s) are present in a region which is single-stranded when the two complementary strands of the double-stranded molecule are annealed (i.e. in a 5′ overhanging portion), then the RNase will be an endonuclease which has specificity for single strands containing ribonucleotides. For cleavage with ribonuclease it is preferred to include two or more consecutive ribonucleotides, and preferably from 2 to 10 or from 5 to 10 consecutive ribonucleotides. The precise sequence of the ribonucleotides is generally not material, except that certain RNases have specificity for cleavage after certain residues. Suitable RNases include, for example, RNaseA, which cleaves after C and U residues. Hence, when cleaving with RNaseA the cleavage site must include at least one ribonucleotide which is C or U.

Polynucleotides incorporating one or more ribonucleotides can be readily synthesised using standard techniques for oligonucleotide chemical synthesis with appropriate ribonucleotide precursors. If the double-stranded nucleic acid molecule is prepared by solid-phase nucleic acid amplification, then it is convenient to incorporate one or more ribonucleotides into one of the primers to be used for the amplification reaction.

### iv) Photochemical Cleavage

The term “photochemical cleavage” encompasses any method which utilises light energy in order to achieve cleavage of one or both strands of the double-stranded nucleic acid molecule.

A site for photochemical cleavage can be provided by a non-nucleotide chemical spacer unit in one of the strands of the double-stranded molecule (or the amplification primer from which this strand is derived if prepared by solid-phase amplification). Suitable photochemical cleavable spacers include the PC spacer phosphoamidite (4-(4,4′-Dimethoxytrityloxy)butyramidomethyl)-1-(2-nitrophenyl)-ethyl]-2-cyanoethyl-(N,N-diisopropyl)-phosphoramidite) supplied by Glen Research, Sterling, Va., USA (cat number 10-4913-XX), which can be cleaved by exposure to a UV light source.

This spacer unit can be attached to the 5′ end of a polynucleotide, together with a thiophosphate group which permits attachment to a solid surface, using standard techniques for chemical synthesis of oligonucleotides. Conveniently, this spacer unit can be incorporated into a forward or reverse amplification primer to be used for synthesis of a photocleavable double-stranded nucleic acid molecule by solid-phase amplification.

### v) Cleavage of Hemimethylated DNA

Site-specific cleavage of one strand of a double-stranded nucleic acid molecule may also be achieved by incorporating one or more methylated nucleotides into this strand and then cleaving with an endonuclease enzyme specific for a recognition sequence including the methylated nucleotide(s).

The methylated nucleotide(s) will typically be incorporated in a region of one strand of the double-stranded nucleic acid molecule having a complementary stretch of non-methylated deoxyribonucleotides on the complementary strand, such that annealing of the two strands produces a hemimethylated duplex structure. The hemimethylated duplex may then be cleaved by the action of a suitable endonuclease. For the avoidance of doubt, enzymes which cleave such hemimethylated target sequences are not to be considered as “restriction endonucleases” excluded from the scope of the second aspect of the invention, but rather are intended to form part of the subject-matter of the invention.

Polynucleotides incorporating one or methylated nucleotides may be prepared using standard techniques for automated DNA synthesis, using appropriately methylated nucleotide precursors. If the double-stranded nucleic acid molecule is prepared by solid-phase nucleic acid amplification, then it is convenient to incorporate one or more methylated nucleotides into one of the primers to be used for the amplification reaction.

### vi) PCR Stoppers

In another embodiment of the invention the double-stranded nucleic acid may be prepared by solid-phase amplification using forward and reverse primers, one of which contains a “PCR stopper”. A “PCR stopper” is any moiety (nucleotide or non-nucleotide) which prevents read-through of the polymerase used for amplification, such that it cannot copy beyond that point. The result is that amplified strands derived by extension of the primer containing the PCR stopper will contain a 5′ overhanging portion. This 5′ overhang (other than the PCR stopper itself) may be comprised of naturally occurring deoxyribonucleotides, with predominantly natural backbone linkages, i.e. it may simply be a stretch of single-stranded DNA. The molecule may then be cleaved in the 5′ overhanging region with the use of a cleavage reagent (e.g. an enzyme) which is selective for cleavage of single-stranded DNA but not double stranded DNA, for example mung bean nuclease.

The PCR stopper may be essentially any moiety which prevents read-through of the polymerase to be used for the amplification reaction. Suitable PCR stoppers include, but are not limited to, hexaethylene glycol (HEG), abasic sites, and any non-natural or modified nucleotide which prevents read-through of the polymerase, including DNA analogues such as peptide nucleic acid (PNA).

Stable abasic sites can be introduced during chemical oligonucleotide synthesis using appropriate spacer units containing the stable abasic site. By way of example, abasic furan (5′-O-Dimethoxytrityl-1′,2′-Dideoxyribose-3′-[(2-cyanoethyl)-(N,N-diisopropyl)]-phosphoramidite) spacers commercially available from Glen Research, Sterling, Va., USA, can be incorporated during chemical oligonucleotide synthesis in order to introduce an abasic site. Such a site can thus readily be introduced into an oligonucleotide primer to be used in solid-phase amplification. If an abasic site is incorporated into either forward or reverse amplification primer the resulting amplification product will have a 5′ overhang on one strand which will include the abasic site (in single-stranded form). The single-stranded abasic site may then be cleaved by the action of a suitable chemical agent (e.g. exposure to alkali) or an enzyme (e.g. AP-endonuclease VI, Shida et al. Nucleic Acids Research, 1996, Vol. 24, 4572-4576).

vii) Cleavage of Peptide Linker

A cleavage site can also be introduced into one strand of the double-stranded nucleic molecule by preparing a conjugate structure in which a peptide molecule is linked to one strand of the nucleic acid molecule (or the amplification primer from which this strand is derived if prepared by solid-phase amplification). The peptide molecule can subsequently be cleaved by a peptidase enzyme of the appropriate specificity, or any other suitable means of non-enzymatic chemical or photochemical cleavage. Typically, the conjugate between peptide and nucleic acid will be formed by covalently linking a peptide to one strand only of the double-stranded nucleic acid molecule, with the peptide portion being conjugated to the 5′ end of this strand, adjacent to the point of attachment to the solid surface. If the double-stranded nucleic acid is prepared by solid-phase amplification, the peptide conjugate may be incorporated at the 5′ end of one of the amplification primers. Obviously the peptide component of this primer will not be copied during PCR amplification, hence the “bridged” amplification product will include a cleavable 5′ peptide “overhang” on one strand.

Conjugates between peptides and nucleic acids wherein the peptide is conjugated to the 5′ end of the nucleic acid can be prepared using techniques generally known in the art. In one such technique the peptide and nucleic acid components of the desired amino acid and nucleotide sequence can be synthesised separately, e.g. by standard automated chemical synthesis techniques, and then conjugated in aqueous/organic solution. By way of example, the OPeC™ system commercially available from Glen Research is based on the “native ligation” of an N-terminal thioester-functionalized peptide to a 5′-cysteinyl oligonucleotide. Pentafluorophenyl S-benzylthiosuccinate is used in the final coupling step in standard Fmoc-based solid-phase peptide assembly. Deprotection with trifluoroacetic acid generates, in solution, peptides substituted with an N-terminal S-benzylthiosuccinyl group. O-trans-4-(N-a-Fmoc-S-tert-butylsulfenyl-1-cysteinyl)aminocyclohexyl O-2-cyanoethyl-N,N-diisopropylphosphoramidite is used in the final coupling step in standard phosphoramidite solid-phase oligonucleotide assembly. Deprotection with aqueous ammonia solution generates in solution 5′-S-tert-butylsulfenyl-L-cysteinyl functionalized oligonucleotides. The thiobenzyl terminus of the Modified Peptide is converted to the thiophenyl analogue by the use of thiophenol, whilst the Modified Oligonucleotide is reduced using the tris(carboxyethyl)phosphine. Coupling of these two intermediates, followed by the “native ligation” step, leads to formation of the Oligonucleotide-Peptide Conjugate.

The conjugate strand containing peptide and nucleic acid can be covalently attached to a solid support using any suitable covalent linkage technique known in the art which is compatible with the chosen surface. If the peptide/nucleic acid conjugate structure is an amplification primer to be used for solid-phase PCR amplification, attachment to the solid support must leave the 3′ end of the nucleic acid component free.

The peptide component can be designed to be cleavable by any chosen peptidase enzyme, of which many are known in the art. The nature of the peptidase is not particularly limited, it is necessary only for the peptidase to cleave somewhere in the peptide component. Similarly, the length and amino acid sequence of the peptide component is not particularly limited except by the need to be “cleavable” by the chosen peptidase.

The length and precise sequence of the nucleic acid component is also not particularly limited, it may be of any desired sequence. If the nucleic acid component is to function as a primer in solid-phase PCR, then its length and nucleotide sequence will be selected to enable annealing to the template to be amplified.

In order to generated a linearised template suitable for sequencing it is necessary to remove “unequal” amounts of the complementary strands in the bridged structure formed by amplification so as to leave behind a linearised template for sequencing which is fully or partially single stranded. Thus, if amplification is performed using identical forward and reverse amplification primers (i.e. a single primer amplification) then in order to prepare templates for sequencing it may be necessary to linearise by cleavage at a site in the template which is not derived from the amplification primers. Otherwise, if all primers were to contain an identical cleavage site, cleavage would remove equal portions of both strands in the bridged structure and would not generate a linearised template for sequencing which is fully or partially single stranded. If a single “universal” primer is used, for example, to amplify templates comprising target nucleic sequences modified by the addition of common universal adaptor sequences, it may be possible to cleave at a cleavage site within the target sequence in order to linearise the bridged amplification products.

Following the cleavage step, regardless of the method used for cleavage, the product of the cleavage reaction may be subjected to denaturing conditions in order to remove the portion(s) of the cleaved strand(s) that are not attached to the solid support. Suitable denaturing conditions will be apparent to the skilled reader with reference to standard molecular biology protocols (Sambrook et al., 2001, Molecular Cloning, A Laboratory Manual, 3rd Ed, Cold Spring Harbor Laboratory Press, Cold Spring Harbor Laboratory Press, NY; Current Protocols, eds Ausubel et al.).

Denaturation (and subsequent re-annealing of the cleaved strands) results in the production of a sequencing template which is partially or substantially single-stranded. A sequencing reaction may then be initiated by hybridisation of a sequencing primer to the single-stranded portion of the template.

Thus, the invention encompasses methods wherein the nucleic acid sequencing reaction comprises hybridising a sequencing primer to a single-stranded region of a linearised amplification product, sequentially incorporating one or more nucleotides into a polynucleotide strand complementary to the region of amplified template strand to be sequenced, identifying the base present in one or more of the incorporated nucleotide(s) and thereby determining the sequence of a region of the template strand.

One preferred sequencing method which can be used in accordance with the invention relies on the use of modified nucleotides that can act as chain terminators. Once the modified nucleotide has been incorporated into the growing polynucleotide chain complementary to the region of the template being sequenced there is no free 3′-OH group available to direct further sequence extension and therefore the polymerase can not add further nucleotides. Once the nature of the base incorporated into the growing chain has been determined, the 3′ block may be removed to allow addition of the next successive nucleotide. By ordering the products derived using these modified nucleotides it is possible to deduce the DNA sequence of the DNA template. Such reactions can be done in a single experiment if each of the modified nucleotides has attached a different label, known to correspond to the particular base, to facilitate discrimination between the bases added at each incorporation step. Alternatively, a separate reaction may be carried out containing each of the modified nucleotides separately.

The modified nucleotides may carry a label to facilitate their detection. Preferably this is a fluorescent label. Each nucleotide type may carry a different fluorescent label. However the detectable label need not be a fluorescent label. Any label can be used which allows the detection of an incorporated nucleotide.

One method for detecting fluorescently labelled nucleotides comprises using laser light of a wavelength specific for the labelled nucleotides, or the use of other suitable sources of illumination. The fluorescence from the label on the nucleotide may be detected by a CCD camera or other suitable detection means.

The methods of the invention are not limited to use of the sequencing method outlined above, but can be used in conjunction with essentially any sequencing methodology which relies on successive incorporation of nucleotides into a polynucleotide chain. Suitable techniques include, for example, Pyrosequencing™, FISSEQ (fluorescent in situ sequencing), MPSS (massively parallel signature sequencing) and sequencing by ligation-based methods.

The target polynucleotide to be sequenced using the method of the invention may be any polynucleotide that it is desired to sequence. The target polynucleotide may be of known, unknown or partially known sequence, for example in re-sequencing applications. Using the solid phase amplification method described in detail herein it is possible to prepare templates for sequencing starting from essentially any double-stranded target polynucleotide of known, unknown or partially known sequence. With the use of arrays it is possible to sequence multiple targets of the same or different sequence in parallel.

The invention will be further understood with reference to the following experimental examples.

### EXAMPLE 1

### Formation of Templates for Solid-Phase Amplification

Templates for solid-phase amplification were first prepared by standard solution phase PCR.

The following oligonucleotide primers were used for template preparation:

A + AlbFor (SEQ ID NO:7):
5′-gctggcacgtccgaacgcttcgttaatccgttgaggatcagctgaag
acggtgat
B + Sbs2back (SEQ ID NO:8):
5′-cgtcgtctgccatggcgcttcggtggatatgaactgatgaaggtata
gatatagag
C + Sbs2back (SEQ ID NO:9):
5′-acggccgctaatatcaacgcgtcgaatccgcaactgatgaaggtata
gatatagag
D + Sbs2back (SEQ ID NO:10):
5′-tgccgcgttacgttagccggactattcgatgcagcgatgaaggtata
gatatagag

Separate solution phase PCR reactions were set up using primer pairs A+AlbFor/B+Sbs2back, A+AlbFor/C+Sbs2back, or A+AlbFor/D+Sbs2back and a template containing 20 nt of rat albumin sequence. The PCR reactions contained 0.5 μM each primer and 4 pM template in a 50 μl JumpStart RedTaq (Sigma) PCR reaction. Reactions were put through 30 cycles of thermal cycling with an annealing temperature of 55° C. Reaction products were analysed by gel electrophoresis, and were judged to contain a single product of the expected size. The PCR reactions were therefore purified through a standard PCR purification kit (QIAGEN) prior to use as templates for cluster formation.

For the LongP5/LongP7 primer pair, the albumin template as it stands could be used for cluster formation.

### Solid-Phase Amplification

The following oligonucleotide primers were prepared with 5′ thiophosphate modifications to allow covalent attachment to a solid-supported hydrogel surface:

A:

(SEQ ID NO:11)

5′-ttttttttttgctggcacgtccgaacgcttcgttaatccgttga
g-3′
B:

(SEQ ID NO:12)

5′-ttttttttttcgtcgtctgccatggcgcttcggtggatatgaac
t-3′
C:

(SEQ ID NO:13)

5′-ttttttttttacggccgctaatatcaacgcgtcgaatccgcaac
t-3′
D:

(SEQ ID NO:14)

5′-tttttttttttgccgcgttacgttagccggactattcgatgcag
c-3′
LongP5:

(SEQ ID NO:1)

5′-ttttttttttcgaattcactagtgattaatgatacggcgaccaccg
a-3′
LongP7:

(SEQ ID NO:16)

5′-ttttttttttgcgggaattcgattcaagcagaagacggcatacg
a-3′

Solid-phase amplification was carried out in 8 channel glass chips coated with a polyacrylamide hydrogel, as follows.

The solid supports used in this experiment were 8-channel glass chips such as those provided by Micronit (Twente, Nederland) or IMT (Neuchâtel, Switzerland). However, the experimental conditions and procedures are readily applicable to other solid supports.

Chips were washed as follows: neat Decon for 30 min, milliQ H₂O for 30 min, NaOH 1N for 15 min, milliQ H₂O for 30 min, HCl 0.1N for 15 min, milliQ H₂O for 30 min.

### Polymer Solution Preparation

For 10 ml of 2% polymerisation mix.
10 ml of 2% solution of acrylamide in milliQ H2O
165 μl of a 100 mg/ml N-(5-bromoacetamidylpentyl) acrylamide (BRAPA) solution in DMF (23.5 mg in 235 μl DMF)
11.5 μl of TEMED
100 μl of a 50 mg/ml solution of potassium persulfate in milliQ H₂O (20 mg in 400 μl H₂O)

The 10 ml solution of acrylamide was first degassed with argon for 15 min. The solutions of BRAPA, TEMED and potassium persulfate were successively added to the acrylamide solution. The mixture was then quickly vortexed and immediately used. Polymerization was then carried out for 1 h 30 at RT. Afterwards the channels were washed with milliQ H₂O for 30 min. The slide was then dried by flushing argon through the inlets and stored under low pressure in a dessicator.

### Synthesis of N-(5-bromoacetamidylpentyl) Acrylamide (BRAPA)

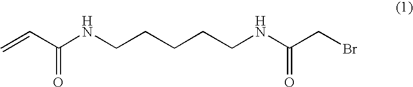

N-Boc-1,5-diaminopentane toluene sulfonic acid was obtained from Novabiochem. The bromoacetyl chloride and acryloyl chloride were obtained from Fluka. All other reagents were Aldrich products.

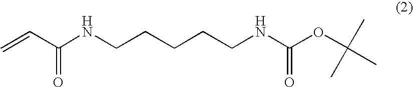

To a stirred suspension of N-Boc-1,5-diaminopentane toluene sulfonic acid (5.2 g, 13.88 mmol) and triethylamine (4.83 ml, 2.5 eq) in THF (120 ml) at 0° C. was added acryloyl chloride (1.13 ml, 1 eq) through a pressure equalized dropping funnel over a one hour period. The reaction mixture was then stirred at room temperature and the progress of the reaction checked by TLC (petroleum ether:ethyl acetate 1:1). After two hours, the salts formed during the reaction were filtered off and the filtrate evaporated to dryness. The residue was purified by flash chromatography (neat petroleum ether followed by a gradient of ethyl acetate up to 60%) to yield 2.56 g (9.98 mmol, 71%) of product 2 as a beige solid. ¹H NMR (400 MHz, d₆-DMSO): 1.20-1.22 (m, 2H, CH₂), 1.29-1.43 (m, 13H, tBu, 2×CH₂), 2.86 (q, 2H, J=6.8 Hz and 12.9 Hz, CH₂), 3.07 (q, 2H, J=6.8 Hz and 12.9 Hz, CH₂), 5.53 (dd, 1H, J=2.3 Hz and 10.1 Hz, CH), 6.05 (dd, 1H, J=2.3 Hz and 17.2 Hz, CH), 6.20 (dd, 1H, J=10.1 Hz and 17.2 Hz, CH), 6.77 (t, 1H, J=5.3 Hz, NH), 8.04 (bs, 1H, NH). Mass (electrospray+) calculated for C₁₃H₂₄N₂O₃ 256, found 279 (256+Na⁺).

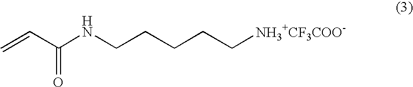

Product 2 (2.56 g, 10 mmol) was dissolved in trifluoroacetic acid:dichloromethane (1:9, 100 ml) and stirred at room temperature. The progress of the reaction was monitored by TLC (dichloromethane:methanol 9:1). On completion, the reaction mixture was evaporated to dryness, the residue co-evaporated three times with toluene and then purified by flash chromatography (neat dichloromethane followed by a gradient of methanol up to 20%). Product 3 was obtained as a white powder (2.43 g, 9 mmol, 906). ¹H NMR (400 MHz, D₂O): 1.29-1.40 (m, 2H, CH₂), 1.52 (quint., 2H, J=7.1 Hz, CH₂), 1.61 (quint., 2H, J=7.7 Hz, CH₂), 2.92 (t, 2H, J=7.6 Hz, CH₂), 3.21 (t, 2H, J=6.8 Hz, CH₂), 5.68 (dd, 1H, J=1.5 Hz and 10.1 Hz, CH), 6.10 (dd, 1H, J=1.5 Hz and 17.2 Hz, CH), 6.20 (dd, 1H, J=10.1 Hz and 17.2 Hz, CH). Mass (electrospray+) calculated for C₈H₁₆N₂O 156, found 179 (156+Na⁺).

To a suspension of product 3 (6.12 g, 22.64 mmol) and triethylamine (6.94 ml, 2.2 eq) in THF (120 ml) was added bromoacetyl chloride (2.07 ml, 1.1 eq), through a pressure equalized dropping funnel, over a one hour period and at −60° C. (cardice and isopropanol bath in a dewar). The reaction mixture was then stirred at room temperature overnight and the completion of the reaction was checked by TLC (dichloromethane:methanol 9:1) the following day. The salts formed during the reaction were filtered off and the reaction mixture evaporated to dryness. The residue was purified by chromatography (neat dichloromethane followed by a gradient of methanol up to 5%). 3.2 g (11.55 mmol, 51%) of the product 1 (BRAPA) were obtained as a white powder. A further recrystallization performed in petroleum ether:ethyl acetate gave 3 g of the product 1. ¹H NMR (400 MHz, d₆-DMSO) :1.21-1.30 (m, 2H, CH₂), 1.34-1.48 (m, 4H, 2×CH₂), 3.02-3.12 (m, 4H, 2×CH₂), 3.81 (s, 2H, CH₂), 5.56 (d, 1H, J=9.85 Hz, CH), 6.07 (d, 1H, J=16.9 Hz, CH), 6.20 (dd, 1H, J=10.1 Hz and 16.9 Hz, CH), 8.07 (bs, 1H, NH), 8.27 (bs, 1H, NH). Mass (electrospray+) calculated for C₁₀H₁₇BrN₂O₂ 276 or 278, found 279 (278+H⁺), 299 (276+Na⁺).

Grafting (covalent attachment) of the 5′-phosphorothioate oligonucleotide primers was carried out using 80 μl of appropriately diluted primer mix per channel in 10 mM phosphate buffer pH7 for 1 h at RT.

The appropriate templates for solid-phase amplification (prepared as described above) were hybridised to the grafted primers immediately prior to the PCR reaction. The PCR reaction thus began with an initial primer extension step rather than template denaturation.

The hybridization procedure began with a heating step in a stringent buffer (95° C. for 5 minutes in TE) to ensure complete denaturation prior to hybridisation of the PCR template. Hybridization was then carried out in 5×SSC, using template diluted to the desired final concentration.

After the hybridization, the chip was washed for 5 minutes with milliQ water to remove salts.

Surface amplification was carried out by thermocycled PCR in an MJ Research thermocycler.

A typical PCR program is as follows:

1—97.5° C. for 0:45

2—X° C. for 1:30

3—73° C. for 1:30

4—Goto 1 [40] times

5—73° C. for 5:00

6—20° C. for 3:00

7—End

Since the first step in the amplification reaction was extension of the primers bound to template in the initial hybridisation step the first denaturation and annealing steps of this program are omitted (i.e. the chip is placed on the heating block only when the PCR mix is pumped through the flow cell and the temperature is at 73° C.).

As with any PCR reaction, the annealing temperature (X° C., step 2) depends on the primer pair that is used. Typical annealing temperatures are in the range of 55-58° C. For any given primer-pair the optimum annealing temperature can be determined by experiment. The number of PCR cycles may be varied if required.

PCR was carried out in a reaction solution comprising 1×PCR reaction buffer (supplied with the enzyme) 1M betain, 1.3% DMSO, 200 μM dNTPs and 0.025 U/μL Taq polymerase.

Following amplification the chips were stained with SyBr Green-I in TE buffer ( 1/10 000), using 100 μl per channel, and the amplified colonies visualised using objective 0.4, Filter Xf 22 and 1 second acquisition time (gain 1).

### Results

A first experiment was carried out in order to assess cluster/colony formation with three different pairs of “long” primers, as compared to a primer-pair of standard length.

An polyacrylamide coated 8 channel glass chip was grafted with the following combinations of the above-described oligonucleotide primers:

Primer pair A/B in channels 1 and 2,

Primer pair A/C in channels 3 and 4,

Primer pair A/D in channels 5 and 6 and

The “standard” P5/P7 primer pair in channels 7 and 8.

Each primer was used at 0.5 μM final concentration in the grafting solution.

After grafting, appropriate templates were hybridised in channels 2, 4, 6 and 8. For the primer-pairs A/B, A/C and A/D the templates prepared by solution-phase PCR as described above were used. For primer-pair P5/P7 the template was a previously prepared construct containing flanking sequences complementary to the P5 and P7 primers.

After template hybridisation, solid-phase amplification was carried out as described above in order to form clusters.

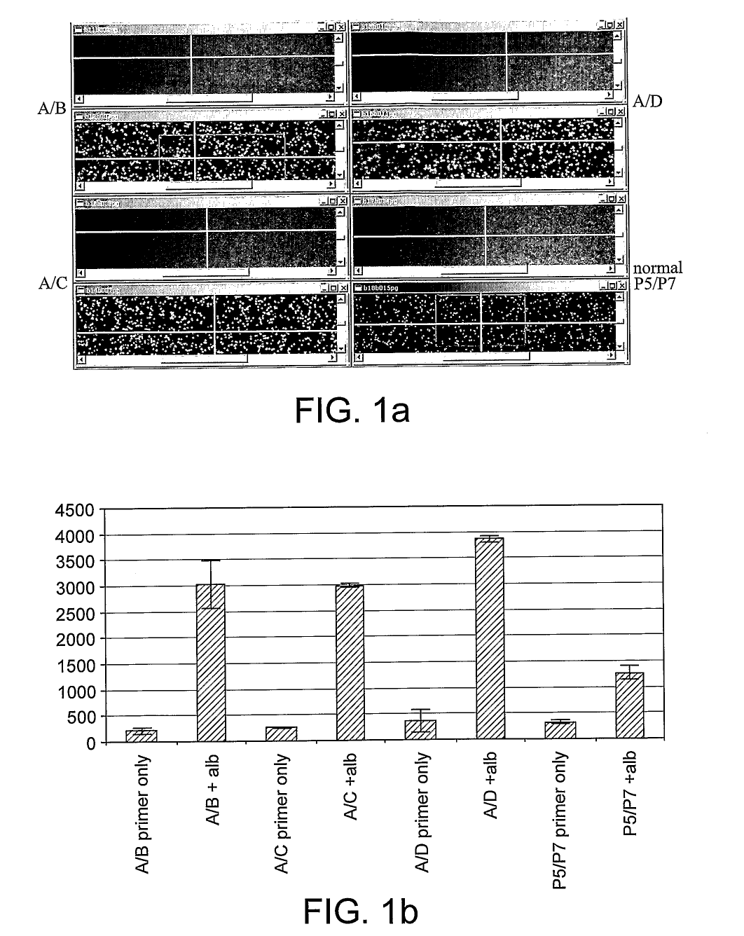

After cluster formation, the chip was stained with SyBr Green and scanned. The results are shown in FIG. 1, and clearly show the formation of more intense, larger clusters with the longer primer pairs (A/B, A/C or A/D), compared to the standard primer pair (P5/P7).

A second experiment was carried out in order to assess cluster formation with “long” P5/P7 primers versus “standard” P5/P7 primers with different amplification templates.

A polyacrylamide coated 8 channel glass chip was grafted using primer pairs P5/P7 in channels 1-4, and long P5/P7 in channels 5-8, following the protocol outlined above. Again, each primer was added to the grafting solution to give a final concentration of 0.5 μM.

After the primer grafting step, templates for amplification were hybridised to the immobilised primers. The following templates were used:

1) A library of PhiX genomic fragments flanked by sequences which permit annealing to the P5/P7 long and short primers. This template was hybridised at 200 pM in channels 1 and 5.

2) A fragment of rat albumin DNA flanked by sequences which permit annealing to the P5/P7 long and short primers. This template was hybridised at 10 pM in channels 2 and 6.

3) A fragment of lambda14 DNA flanked by sequences which permit annealing to the P5/P7 long and short primers. This template was hybridised at 50 pM in channels 3 and 7.

After template hybridisation, solid-phase amplification was carried out in all channels using the protocol outlined above in order to form clusters.

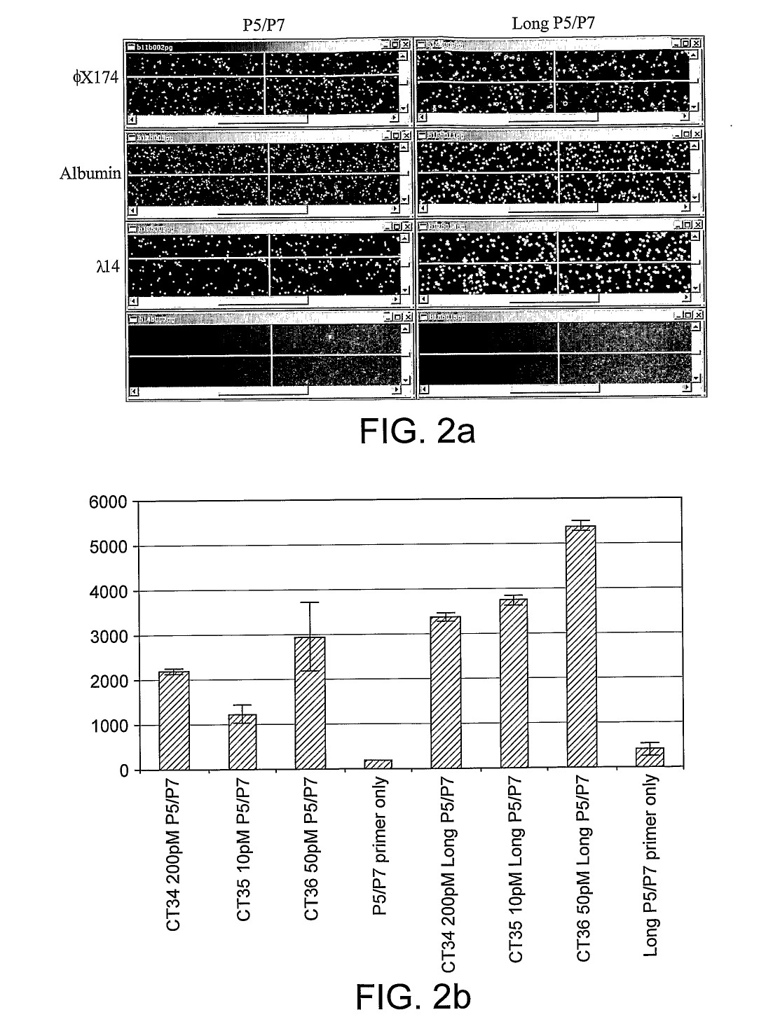

After cluster formation, the chip was stained with SyBr Green and scanned. The results are shown in FIG. 2, and clearly show the formation of more intense, larger clusters with the long P5/P7 primer pair, compared to the standard primer pair.

### EXAMPLE 2

### Cluster Formation by Single Primer Amplification with Long Primers

### Formation of Templates for Solid Phase Amplification

Templates for solid phase amplification were prepared by solution phase PCR according to the method described in example 1 with the following primers:

B+SBS2back (see example 1) and

B+albfor:

5′CGTCGTCTGCCATGGCGCTTCGGTGGATATGAACTGATCAGCTGAAGACGGTGAT (SEQ ID NO:17)

Or,

C+Sbs2back (see example 1) and

C+albfor:

5′ ACGGCCGCTAATATCAACGCGTCGAATCCGCAACTGATCAGCTGAAGACGGTGAT ((SEQ ID NO:18)

Amplification was carried out as described in example 1

### Solid-Phase Amplification

Oligonucleotide primers B or C, described in example 1, were covalently attached to a solid-supported hydrogel surface and used to amplify the corresponding cognate template by solid-phase amplification as described in example 1.

### Results

A polyacrylamide-coated 8 channel glass chip was grafted with the following combinations of the above-described oligonucleotide primers as described in example 1:

Primer B only in channels 1 and 2

Primer pair A/B in channels 3 and 4

Primer C only in channels 5 and 6

Primer pair A/C in channels 7 and 8.

After grafting appropriate templates were hybridised in the following channels:

Template generated by solution phase PCR with primers B+SBS2back and B+albfor: channels 1 and 2;

Template generated by solution phase PCR with primers B+SBS2back and A+albfor: channels 3 and 4;

Template generated by solution phase PCR with primers C+SBS2back and C+albfor: channels 5 and 6;

Template generated by solution phase PCR with primers C+SBS2back and A+albfor: channels 7 and 8.

After template hybridisation, solid-phase amplification was carried out according to example 1 to form clusters.

After cluster formation, the chip was stained as described in example 1 and scanned.

In all channels, clusters were clearly visible. Similar numbers of clusters were observed in all channels, and clusters appeared to have similar intensities. This example illustrates the effectiveness of solid-phase amplification with a single primer.

1. A method of amplifying one or more nucleic acid templates on a solid support which comprises:
a) bringing into contact the following components under conditions which permit a nucleic acid amplification reaction:
i) a solid support,
ii) a plurality of forward and reverse amplification primers, wherein the solid support is provided with the forward and/or reverse amplification primers immobilised thereon, and
iii) one or more nucleic acid templates to be amplified comprising at the 3′ end a primer-binding sequence which is a sequence of nucleotides capable of annealing to the forward amplification primers and at the 5′ end a primer-binding sequence which is a sequence of nucleotides the complement of which is capable of annealing to the reverse amplification primers; and
b) carrying out a nucleic acid amplification reaction whereby said template(s) is/are amplified with said forward and reverse amplification primers,
characterised in that the amplification primers immobilised on the solid support comprise a template-specific portion which is a sequence of at least 26 consecutive nucleotides capable of annealing to a primer binding sequence in the template or the complement thereof and that the forward and reverse primers are not capable of annealing to any part of the template other than their respective primer binding sequences during the nucleic acid amplification reaction.

2. The method according to claim 1 wherein the one or more templates to be amplified each include a target sequence located between the two primer binding sequences, each said target sequence representing a fragment of the full sequence of a nucleic acid sample of interest, and the forward and reverse primers are selected based on knowledge of the full sequence of the nucleic acid sample of interest so as not to be capable of annealing to any part of the template other than their respective primer binding sequences during the nucleic acid amplification reaction.

3. The method according to claim 2 wherein the nucleic acid sample of interest is genomic DNA.

4. The method according to claim 3 wherein the nucleic acid sample of interest is human genomic DNA.

5. The method according to claim 4 wherein the nucleic acid sample of interest represents from 50% to 100% of the complete human genome.

6. The method according to claim 1 wherein the nucleic acid template(s) is/are produced by providing one or more target nucleic acid molecules to be amplified and adding thereto at the 3′ end a first adaptor polynucleotide comprising a primer binding sequence capable of annealing to the forward amplification primers and at the 5′ end a second adaptor polynucleotide comprising a primer binding sequence the complement of which is capable of annealing to the reverse amplification primers.

7. The method according to claim 6 wherein a plurality of templates to be amplified in a single amplification reaction are produced by providing a plurality of target nucleic acid molecules of different sequence and adding thereto at the 3′ end a first universal adaptor polynucleotide comprising a sequence of nucleotides capable of annealing to the forward amplification primers and at the 5′ end a second universal adaptor polynucleotide comprising a sequence of nucleotides the complement of which is capable of annealing to the reverse amplification primers.

8. The method according to claim 7 wherein the plurality of target nucleic acid molecules of different sequence are genomic DNA fragments.

9. The method according to claim 8 wherein the genomic DNA fragments are human genomic DNA fragments.

10. The method according to claim 9 wherein the sequences of the template-specific portions in the forward and reverse amplification primers are selected such that any sequence of 20 consecutive nucleotides in either template-specific portion is at least 2 bases different to any 20-mer in either strand of the human genome.

11. The method according to claim 1 wherein both the forward and reverse amplification primers comprise a template-specific portion which is a sequence of at least 26 consecutive nucleotides.

12. The method according to claim 11 wherein the template-specific portion in the forward and/or reverse amplification primers is a sequence of at least 30 consecutive nucleotides.

13. The method according to claim 12 wherein the template-specific portion in the forward and/or reverse amplification primers is a sequence of at least 35 consecutive nucleotides.

14. The method according to claim 11 wherein the template-specific portion in the forward and/or reverse amplification primers is a sequence of less than 50 consecutive nucleotides.

15. The method according to claim 14 wherein the template-specific portion in the forward and/or reverse amplification primers is a sequence of from 30 to 45 consecutive nucleotides.

16. The method according to claim 15 wherein the template-specific portion in the forward and/or reverse amplification primers is a sequence of from 35 to 40 consecutive nucleotides.

17. The method according to claim 16 wherein the template-specific portion in the forward and/or reverse amplification primers is a sequence of 35 consecutive nucleotides

18. The method according to claim 1 wherein the forward and/or the reverse amplification primers immobilised on the solid support additionally comprise a linker portion which is not capable of annealing to the template to be amplified or the complement thereof.

19. The method according to claim 18 wherein the linker portion is a sequence of nucleotides which is not capable of annealing to the template to be amplified or the complement thereof.

20. The method according to claim 19 wherein the linker portion consists of from 1 to 20 consecutive nucleotides.

21. The method according to claim 20 wherein the linker portion consists of from 1 to 10 consecutive nucleotides

22. The method according to claim 20 wherein the linker portion consists of thymidine nucleotides.

23. The method according to claim 18 wherein the linker portion comprises a non-nucleotide chemical moiety.

24. The method according to claim 1 wherein in step a) the solid support is provided with both the forward and reverse amplification primers immobilised thereon.

25. The method according to claim 24 wherein in step a) the solid support is provided with both the forward and reverse amplification primers and the nucleic acid template to be amplified immobilised thereon, the template being attached to the solid support at the 5′ end.

26. The method according to claim 1 wherein the forward and reverse primers are identical.

27. The method according to claim 1 wherein the solid support is a solid supported polyacrylamide hydrogel.

28. (canceled)

29. The method of nucleic acid sequencing which comprises amplifying one or more nucleic acid templates using a method as defined in claim 1 and carrying out a sequencing reaction to determine the sequence of the whole or a part of at least one amplified nucleic acid strand produced in the amplification reaction.

30. A solid support having immobilised thereon a plurality of forward and/or reverse amplification primers, characterised in that said forward and/or reverse amplification primers comprise a template-specific portion capable of annealing to the template or the complement thereof which is a sequence of at least 26 consecutive nucleotides.

31. The solid support according to claim 30 wherein the template-specific portion in the forward and/or reverse amplification primers is a sequence of at least 30 consecutive nucleotides.

32. The solid support according to claim 31 wherein the template-specific portion in the forward and/or reverse amplification primers is a sequence of at least 35 consecutive nucleotides.

33. The solid support according to claim 30 wherein the template-specific portion in the forward and/or reverse amplification primers is a sequence of less than 50 consecutive nucleotides.

34. The solid support according to claim 31 wherein the template-specific portion in the forward and/or reverse amplification primers is a sequence of from 30 to 45 consecutive nucleotides.

35. The solid support according to claim 34 wherein the template-specific portion in the forward and/or reverse amplification primers is a sequence of from 35 to 40 consecutive nucleotides.

36. The solid support according to claim 35 wherein the template-specific portion in the forward and/or reverse amplification primers is a sequence of 35 consecutive nucleotides

37. The solid support according to claim 30 wherein the forward and/or the reverse amplification primers immobilised on the solid support additionally comprise a linker portion which is not capable of annealing to the template to be amplified or the complement thereof.

38. The solid support according to claim 37 wherein the linker portion is a sequence of nucleotides which is not capable of annealing to the template to be amplified or the complement thereof.

39. The solid support according to claim 38 wherein the linker portion consists of from 1 to 20 consecutive nucleotides.

40. The solid support according to claim 39 wherein the linker portion consists of from 1 to 10 consecutive nucleotides

41. The solid support according to claim 39 wherein the linker portion consists of thymidine nucleotides.

42. The solid support according to claim 37 wherein the linker portion comprises a non-nucleotide chemical moiety.

43. The solid support according to claim 30 wherein the forward and reverse primers are identical.

44. (canceled)
